(19) United States
(12) Patent Application Publication
(10) Pub. No.: US 2006/0135985 A1
(43) Pub. Date: Jun. 22, 2006

(54) Vulnerable plaque modification methods and apparatuses

(76) Inventors: Daniel L. Cox, Palo Alto, CA (US); Jeffrey T. Ellis, San Francisco, CA (US); Jeong S. Lee, Diamond Bar, CA (US); Klaus Kleine, Los Gatos, CA (US); Alan Tannier, Temecula, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/022,548

(22) Filed: Dec. 21, 2004

(51) Int. Cl. A61M 29/00

(52) U.S. Cl. 606/194

(57) ABSTRACT

A method including introducing an expandable body into a blood vessel at a point coextensive with a vulnerable plaque lesion, and expanding the expandable body from a first diameter to a different second diameter sufficient to modify the shape of an inner diameter of the blood vessel at the point coextensive with the lesion without rupturing the lesion. An apparatus including a cannula having a dimension suitable for insertion into a blood vessel and including an expandable body coupled thereto, the expandable body including a first outer diameter suitable for insertion through the blood vessel and a second outer diameter greater than the first diameter and having a maximum dimension to modify the shape of an inner diameter of the blood vessel and retain a same perimeter. A kit including a cannula including an expandable body and a stent. An expandable framework comprising a polymer material. An apparatus including an expandable body.

### FIELD

Transluminal treatment devices and methods.

### BACKGROUND

Thin-capped fibroatheroma (“TFCA”) or vulnerable plaque refers to an atherosclerotic plaque that may develop inside a blood vessel, such as an artery. The typical vulnerable plaque contains a core filled with lipids, cholesterol crystals and cholesterol esters, macrophages, and other cells. The core has a thin fibrous cap (0.05 millimeters (mm) to 0.10 mm thickness). The fibrous cap may become weakened and rupture. When ruptured, the luminal blood becomes exposed to highly thrombogenic material from the core of the vulnerable plaque, which can result in total thrombotic occlusion of the blood vessel.

There is increasing evidence that the propensity of a vulnerable plaque to rupture is related to an activity of matrix metalloproteinases (“MMPs”), largely synthesized by macrophage-derived foam cells. Specifically, MMPs may degrade extracellular matrix proteins, such as Types I and III collagen that are a significant source of fibrous cap structural integrity. Thus, chronic and/or local inflammation, typically a result of monocyte adhesion, in the plaque can lead to destabilization of the vulnerable plaque and acute coronary syndromes (via thrombosis).

Researchers believe that vulnerable plaque is formed in the following way. Fat droplets are absorbed by the blood vessel (e.g., artery), which causes the release of cytokines (proteins) that lead to inflammation. The cytokines make the artery wall sticky, which attracts monocytes (immune system cells). The monocytes squeeze into the artery wall. Once inside, the monocytes turn into macrophages (cells) and begin to soak-up fat droplets. The fat-filled macrophages form a plaque with a thin covering.

Improvements in imaging techniques, such as optical coherence tomography (“OCT”) and intravascular ultrasound (“IVUS”) offer the opportunity to identify a vulnerable plaque. A need exists, however, for effective methods to treat (e.g., remove, immobilize, modify) a vulnerable plaque.

### SUMMARY

In one embodiment, a method is disclosed. The method includes introducing an expandable body such as a balloon into a blood vessel at a point coextensive with a vulnerable plaque lesion. The method also includes expanding the expandable body from a first diameter to a different second diameter sufficient to modify the shape of an inner diameter of the blood vessel at the point coextensive with the lesion without rupturing the lesion. Typically, a vulnerable plaque will tend to modify a lateral cross-sectional shape from generally circular to oblong or non-circular. By modifying the shape of the lumen, stress on the blood vessel tends to be reduced. In one embodiment, the vulnerable plaque lesion may be gently contacted which may cause injury (without rupture) that can induce neointimal tissue growth to support the lesion. In one embodiment, following the modification of the lumen, the expandable body may be removed leaving no extraneous structure. In another embodiment, a stent may be deployed that supports the vulnerable plaque.

In another embodiment, a method includes introducing a catheter comprising an expandable body such as a balloon having a first portion bounded by a second portion and a third portion into a blood vessel comprising a vulnerable plaque lesion. The first portion is introduced at a point coextensive with a vulnerable plaque lesion. The method also includes expanding the second portion and the third portion of the expandable body to a diameter greater than a diameter of the first portion. Representatively, the first portion may expand significantly less than the second or third portion. In another embodiment, the first portion may not expand at inflation pressures necessary to expand the second and third portions. In one embodiment, a support structure such as a stent may be deployed by the expandable body. A stent, for example, may have a length that is longer than a working length of the first portion of the expandable body so that it may overly the second portion and the third portion. In this manner, the second and third portion may be expanded to anchor the stent to the blood vessel at portions proximal and distal to the vulnerable plaque.

In another embodiment, an apparatus is disclosed. The apparatus includes a cannula having a dimension suitable for insertion into a blood vessel and comprising an expandable body coupled thereto. The expandable body includes, for example, a balloon including a first outer diameter suitable for insertion through the blood vessel and a second outer diameter greater than the first diameter and having a maximum dimension to modify the shape of an inner diameter of the blood vessel and retain a same perimeter.

In another embodiment, a kit is disclosed. The kit includes a cannula having a dimension suitable for insertion into a blood vessel and comprising an expandable body coupled thereto, the expandable body comprising a first outer diameter suitable for insertion through the blood vessel and a second outer diameter greater than the first diameter and the second diameter has a maximum dimension to modify the shape of an inner diameter of the blood vessel and retain a same perimeter. The kit also includes a stent having a diameter suitable for deployment on the expandable body through a blood vessel.

In a further embodiment, an apparatus is disclosed. The apparatus includes an expandable framework having an expanded diameter suitable for placement in a blood vessel and comprising of a first end and a second end and a polymeric material disposed between the first end and the second end and defining a lumen therethrough. The apparatus as a stent may include a metal frame, such as proximal and distal metal end rings of struts with polymeric material formed between the framework. The polymeric material may be formed into struts or suspension elements or may be a mesh or weave wrapped around the metal framework. In another embodiment, the polymeric material may be impregnated or coated with a drug or a cellular component.

In a further embodiment, an apparatus is disclosed. The apparatus includes an expandable body such as a balloon of a catheter assembly having a diameter suitable for insertion into a blood vessel. The expandable body is capable of being modified from a first diameter to a second larger diameter in response to an inflation pressure less than two atmospheres. Following modification, the expandable body has a property such that it becomes non-compliant at an increased inflation pressure less than two atmospheres.

In a still further embodiment, an apparatus is disclosed. The apparatus includes an expandable body such as a balloon of a catheter assembly having a diameter suitable for insertion into a blood vessel. The expandable body is capable of being modified from a first diameter to a second larger diameter that is less than an interior diameter of a target blood vessel. Following modification, the expandable body has a property such that it becomes non-compliant at an increased inflation pressure.

### BRIEF DESCRIPTION OF THE DRAWINGS

FIG. 1 shows a cross-sectional schematic side view of a blood vessel including a vulnerable plaque.

FIG. 2 shows a cross-sectional view of the blood vessel of FIG. 1 through line 1-1′.

FIG. 3 shows a cross-sectional view of the blood vessel of FIG. 1 through line 1-1′ following the modification of the blood vessel lumen into a shape approaching a circular cross section.

FIG. 4 shows a cross-sectional schematic side view of a blood vessel including a vulnerable plaque and a first balloon positioned downstream of the vulnerable plaque in an inflated state.

FIG. 5 shows the blood vessel of FIG. 4 following the introduction of a contrast agent upstream of the first balloon and at the vulnerable plaque.

FIG. 6 shows the blood vessel of FIG. 4 following the introduction of a second balloon at a region in the blood vessel including (coextensive with) the vulnerable plaque.

FIG. 7 shows a cross-sectional side view of the blood vessel of FIG. 6 through line 6-6′.

FIG. 8 shows the blood vessel of FIG. 4 following the inflation of the second balloon to a diameter sufficient to modify a shape of a lumen of the blood vessel into that approaching a circle.

FIG. 9 shows the blood vessel of FIG. 8 through line 8-8′.

FIG. 10 shows a cross-sectional schematic side view of a blood vessel having a catheter assembly including a first balloon and a second balloon introduced therein and including a stent on the second balloon and contrast agent introduced upstream of the first balloon.

FIG. 11 shows the blood vessel of FIG. 10 through line 10-10′.

FIG. 12 shows the blood vessel of FIG. 10 following the inflation of the second balloon.

FIG. 13 shows the blood vessel of FIG. 12 through line 12-12′.

FIG. 14 shows a cross-sectional schematic side view of a blood vessel including a vulnerable plaque and having a catheter assembly introduced having a balloon with a working length longer than the vulnerable plaque such that a portion of the balloon extends downstream of the vulnerable plaque and including a stent on the balloon.

FIG. 15 shows the blood vessel of FIG. 14 following the expansion of a distal portion of the balloon and the introduction of contrast agent into the blood vessel.

FIG. 16 shows the blood vessel of FIG. 15 following the further expansion of the balloon to a point that minimizes the contrast agent around the vulnerable plaque.

FIG. 17 shows a cross-sectional schematic side view of a blood vessel including a vulnerable plaque and having a balloon disposed in the blood vessel with a working length extending downstream of a location including the vulnerable plaque and a stent disposed on the balloon.

FIG. 18 shows a cross-sectional side view of the catheter assembly and stent of FIG. 17 through line 17-17′.

FIG. 19 shows a flatten version of an embodiment of the stent of the catheter assembly of FIG. 17.

FIG. 20 shows the blood vessel of FIG. 17 following the expansion of a distal portion of the balloon of the catheter assembly and the introduction of contrast agent.

FIG. 21 shows the blood vessel of FIG. 20 following the further expansion of the balloon to a point that minimizes the contrast agent around the vulnerable plaque.

FIG. 22 shows the blood vessel of FIG. 21 through line 21-21′.

FIG. 23 shows a cross-sectional schematic side view of an embodiment of a catheter assembly including a balloon (shown inflated) having multiple (two) inflation diameter portions.

FIG. 24 shows a graphical representation of the compliance of different portions of the balloon of the catheter assembly of FIG. 23.

FIG. 25 shows a cross-sectional schematic side view of a portion of a blood vessel including the catheter assembly of FIG. 23 where one portion of the balloon is inflated at a position downstream of a vulnerable plaque and after the introduction of contrast agent into the blood vessel.

FIG. 26 shows the blood vessel of FIG. 25 following the inflation of a second portion of the balloon of the catheter assembly of FIG. 23.

FIG. 27 shows a cross-sectional schematic side view of a blood vessel including a vulnerable plaque and shows another embodiment of a catheter assembly including a balloon having multiple (three) inflation diameter portions including diameters equivalent to the inner diameter of the blood vessel at position upstream and downstream of a vulnerable plaque and a stent on the balloon.

FIG. 28 shows a cross-sectional schematic side view of a distal portion of a catheter assembly including multiple (three) inflation diameter portions and separate inflation cannulas for each portion.

FIG. 29 shows a flattened schematic top view of an embodiment of a portion of a stent that may be suitable for use in conjunction with a catheter assembly of FIG. 27 or FIG. 28.

FIG. 30 shows a flattened schematic top view of a second embodiment of a portion of a stent that may be suitable for use in conjunction with a catheter assembly of FIG. 27 or FIG. 28.

FIG. 31 shows a flattened schematic top view of a third embodiment of a portion of a stent that may be suitable for use in conjunction with a catheter assembly of FIG. 27 or FIG. 28.

FIG. 32 shows a flattened schematic top view of a fourth embodiment of a portion of a stent that may be suitable for use in conjunction with a catheter assembly of FIG. 27 or FIG. 28.

FIG. 33 shows a flattened schematic top view of a fifth embodiment of a portion of a stent that may be suitable for use in conjunction with a catheter assembly of FIG. 27 or FIG. 28.

FIG. 34 shows a flattened schematic top view of a sixth embodiment of a portion of a stent that may be suitable for use in conjunction with a catheter assembly of FIG. 27 or FIG. 28.

FIG. 35 shows a flattened schematic top view of a seventh embodiment of a portion of a stent that may be suitable for use in conjunction with a catheter assembly of FIG. 27 or FIG. 28.

FIG. 36 shows a flattened schematic top view of an eighth embodiment of a portion of a stent that may be suitable for use in conjunction with a catheter assembly of FIG. 27 or FIG. 28.

FIG. 37 shows a flattened schematic top view of a ninth embodiment of a portion of a stent that may be suitable for use in conjunction with a catheter assembly of FIG. 27 or FIG. 28.

FIG. 38 shows a flattened schematic top view of a tenth embodiment of a portion of a stent that may be suitable for use in conjunction with a catheter assembly of FIG. 27 or FIG. 28.

FIG. 39 shows a cross-sectional schematic side view of a blood vessel including a vulnerable plaque and showing a portion of a catheter assembly disposed therein, the catheter assembly including a balloon portion extending from a position upstream to a position downstream of the vulnerable plaque and a stent disposed on the balloon.

FIG. 40 shows the blood vessel of FIG. 39 following a partial expansion of the balloon of the catheter assembly.

FIG. 41 shows the blood vessel of FIG. 40 following the further inflation of the balloon of the catheter assembly.

FIG. 42 shows a flattened top view of an embodiment of a portion of a stent suitable for use with the catheter assembly described with reference to FIGS. 39-41.

FIG. 43 shows a flattened schematic top view of a second embodiment of a portion of a stent suitable for use with the catheter assembly described with reference to FIGS. 39-41.

FIG. 44 shows a cross-sectional schematic side view of a catheter assembly including a spiral balloon.

FIG. 45 shows a cross-sectional schematic side view of a blood vessel including a vulnerable plaque and having the catheter assembly of FIG. 44 disposed in the blood vessel with spirals of the balloon on upstream and downstream sides of the vulnerable plaque.

FIG. 46 shows a flattened schematic top view of a metal-polymer hybrid stent.

FIG. 47 shows a cross-sectional schematic side view of a blood vessel including a vulnerable plaque and having the stent of FIG. 47 disposed therein.

FIG. 48 shows a flattened schematic top view of a stent having a metal frame and a polymer mesh over the frame.

FIG. 49 is a graphical representation of inflation pressure versus balloon diameter for an embodiment of an inflation balloon and a conventional inflation balloon.

FIG. 50 is a graphical representation of inflation pressure versus balloon diameter for an embodiment of an inflation balloon and a conventional inflation balloon.

FIG. 51 shows a schematic side view of a balloon in an inflated state having a dog-bone or dumb-bell shape.

### DETAILED DESCRIPTION

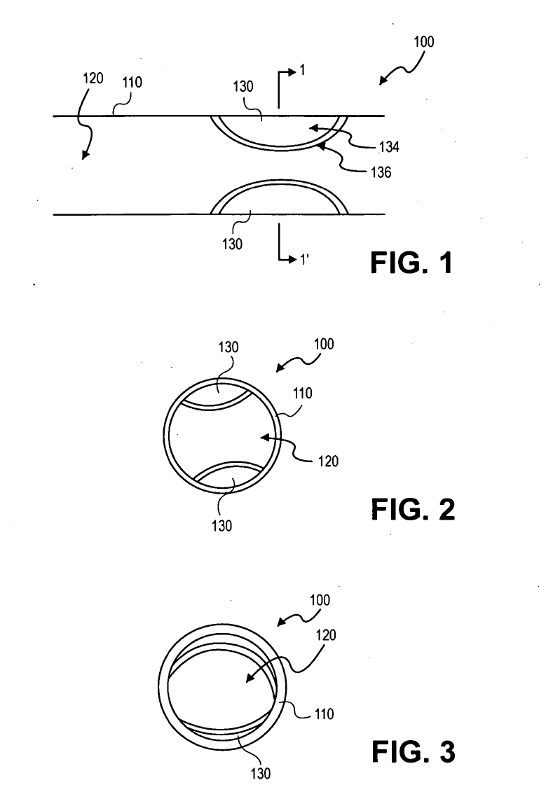

FIG. 1 shows a cross-sectional side view of a portion of a blood vessel, such as a coronary artery. Blood vessel 100 includes vessel wall 110 defining lumen 120 therethrough. Formed within lumen 120 of blood vessel 100 is vulnerable plaque 130. Vulnerable plaque 130 includes core 134 surrounded by fibrous cap 136. Core 134 typically includes lipids, cholesterol crystals, cholesterol esters, macrophages, and other cells. Core material 134 is highly thrombogenic and only fibrous cap 136 prevents release of the thrombogenic materials.

FIG. 2 shows a cross-sectional side view of blood vessel 100 through line 1-1′ of FIG. 1. FIG. 2 shows that a build-up of a lesion or vulnerable plaque 130 extends into the generally circular diameter of lumen 120 of blood vessel 100 and therefore modifies the diameter of lumen 120 from a generally circular shape to an oblong or irregular shape (render an area of the lumen defined by the cross-section other than circular). It is appreciated that the depiction of vulnerable plaque 130 is only an example and that a vulnerable plaque may modify a blood vessel lumen in many ways and occlude the lumen to a greater or lesser extent.

In addition, in response to the build-up of vulnerable plaque 130, a blood vessel such as blood vessel 100 tends to expand to maintain blood flow through the vessel. The expansion of the blood vessel causes the blood vessel to become oblong or non-circular. It is believed that one way to reduce stress on blood vessel 100 and vulnerable plaque 130 within the blood vessel is to reshape the cross-section of the blood vessel to a shape that is generally circular (a generally circular cross-sectional area). However, stretching a blood vessel tends to introduce stress on the vessel or the vulnerable plaque. Therefore, one target to reduce the stress in a blood vessel at an area containing a vulnerable plaque is to make a cross-section of a lumen of the blood vessel circular or approaching a circle in a manner that retains the same lumen perimeter without stretching and possibly rupturing the fibrous cap of the vulnerable plaque.

Modification of a shape of a blood vessel lumen including a vulnerable plaque may be distinguished from a typical angioplasty procedure to treat a stable plaque. A typical angioplasty procedure imparts sufficient force on a lumen and a stable plaque to stretch a blood vessel, forcing a widening of the blood vessel. The widening of the blood vessel may cause undesirable injury which could lead to restenosis. Anti-proliferic drugs are commonly used to inhibit endothelial tissue growth in the region.

A stable plaque generally has a similar fibrous consistency throughout, compared to a vulnerable plaque that is typically patent with a core protected by a fibrous cap. Angioplasty procedures are performed on stable plaques and also performed following the rupture of a vulnerable plaque when the plaque material (e.g., lipid core) leads to occlusions or unstable angina. One target of the reshaping described herein with respect to intact vulnerable plaque is to re-shape a lumen without stretching the blood vessel. Another target is to re-shape a lumen without rupturing the vulnerable plaque.

FIG. 3 shows a cross-sectional side view of blood vessel 100 through line 1-1′ of FIG. 1 following the modification of lumen 120 of blood vessel 100 from the irregular lumen shape shown in FIG. 2 to a shape approaching a circle.

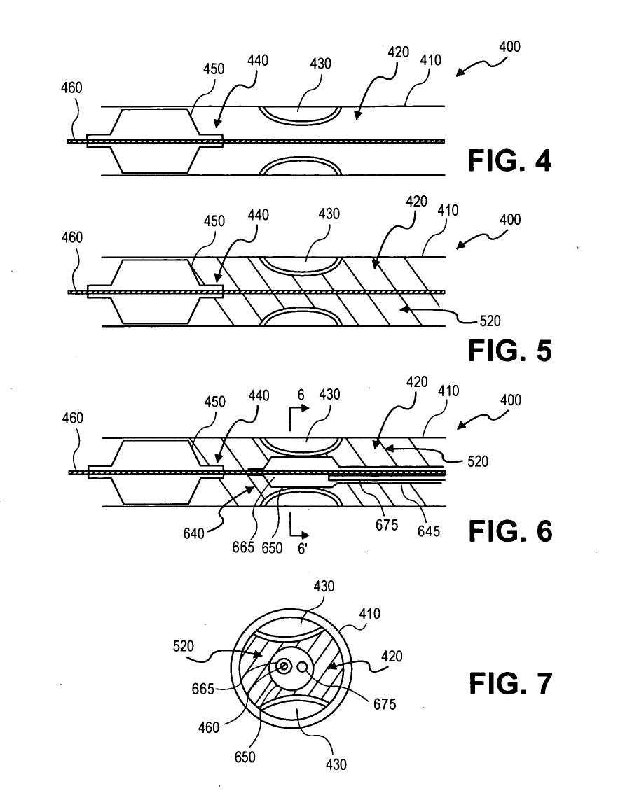

FIGS. 4-9 illustrate one technique for modifying a lumen of a blood vessel containing a vulnerable plaque. FIG. 4 shows blood vessel 400 defined by vessel wall 410 and lumen 420. Vulnerable plaque 430 forms within blood vessel 400 and modifies the shape of lumen 420 from a generally circular shape to an oblong or irregular shape. Vulnerable plaque 430 may be identified using identification technique such as IVUS or OCT.

FIG. 4 shows catheter assembly 440 within lumen 420. Catheter assembly 440 may be introduced into lumen 420 through a guide catheter (not shown). Representatively, a guide catheter having a lumen with an inside diameter suitable to accommodate a distal portion of catheter assembly may initially be introduced through a femoral or radial artery to a point proximal to the region of interest or treatment site. In the example where the region of interest or treatment site is a coronary artery, the guide catheter may be introduced, for example, over guidewire 460 to the ostium of the aorta. Following the introduction of the guide catheter, catheter assembly 440 may be introduced through a lumen of the guide catheter.

Referring to FIG. 4, in one embodiment, catheter assembly 440 includes guidewire 460 (possibly previously introduced) having inflatable balloon 450 at a distal end. An inflation fluid may be introduced through the guidewire to inflate balloon 450. One such guidewire balloon configuration is a PERCUSURG™ catheter assembly, commercially available from Medtronic, Inc. of Minneapolis, Minn. In the embodiment shown in FIG. 4, balloon 450 is introduced to a position downstream of vulnerable plaque 430. FIG. 4 shows balloon 450 in an expanded or inflated state having a diameter substantially equivalent to a diameter of lumen 420 at its target position. In this state, balloon 450 will occlude flow (e.g., blood flow) through lumen 420 of blood vessel 400. Balloon 450 is selectively deflatable to return to a collapsed configuration or a deflated profile.

Following the placement of balloon 450 at a target position downstream of vulnerable plaque 430, a contrast agent may be introduced into blood vessel 400. FIG. 5 shows blood vessel 400 including contrast agent 520 introduced in lumen 420 of the blood vessel and tending to pool, due to the flow restriction caused by balloon 450, around vulnerable plaque 430. Contrast agent 520 may be introduced (e.g., injected) through the previously introduced guide catheter. In FIG. 5, contrast agent 520 is shown as hatching within lumen 420. A similar representation will be used throughout this document.

In FIGS. 4-9, angiographic techniques may be used to assess the circularity of lumen 420 at a position including vulnerable plaque 430. In one embodiment, contrast agent 520 is a radiopaque material such as a diatrizoate such as RENOGRAFIN™ (Bracco Diagnostics, Inc. of Princeton, N.J.) that may be detected using x-ray.

FIG. 6 shows blood vessel 400 following the introduction of catheter assembly 640 in lumen 420. Catheter assembly 640 includes balloon portion 650 at a distal end. A proximal end (proximal skirt) of balloon 650 is connected (e.g., thermally-bonded or glued) to primary cannula 645. Primary cannula 645 extends, in one embodiment, from a proximal end of catheter assembly 640 (e.g., outside a patient) to a region of interest or a treatment site defined by the location of vulnerable plaque 430 and the location of balloon 650. Catheter assembly 640 also includes cannula 660 disposed within a lumen of primary cannula 645. Cannula 660 has a lumen therethrough that may extend through catheter assembly 640 from a proximal port located external to a patient during a treatment procedure to a distal end or exit port terminating within balloon 650. Thus, balloon 650 may be inflated by introducing a fluid through a lumen of cannula 660. Balloon 650 can be selectively inflated by supplying a fluid (e.g., liquid) into a lumen of inflation cannula 660 at a predetermined rate of pressure. Likewise, balloon 650 is selectively deflatable to return to a collapsed configuration or a deflated profile.

Catheter assembly 640 also includes guidewire cannula 665 extending through primary cannula 645 and balloon 650 to a distal end of catheter assembly 640. Guidewire cannula 665 has a lumen therethrough that allows catheter assembly 640 to be fed and maneuvered over guidewire 460 (the same guidewire used in deploying balloon 450). In one embodiment, guidewire cannula 665 extends a length of catheter assembly 640 from a proximal portion intended to be external to a patient during a procedure to a distal end. Representatively, in a typical procedure, guidewire 460 is placed so that balloon 450 is at a desired location in a blood vessel (in this case, downstream of a region of interest or a treatment site including vulnerable plaque 630). Catheter assembly 640 is advanced, possibly through a guide catheter, on/over guidewire 460 to or through a region of interest in an over the wire (OTW) fashion.

FIG. 7 shows a cross-sectional side view through line 6-6′ of FIG. 6. FIG. 7 shows the oblong or non-circular shape of lumen 420 due to the presence of vulnerable plaque 430. Catheter assembly 640 is shown within lumen 420 and balloon 650 is shown in a deflated or partially inflated state such that balloon 650 is not in contact with the fibrous cap of vulnerable plaque 430 or wall 410 of blood vessel 400. FIG. 7 also shows contrast agent 520 surrounding balloon 650 at a location in blood vessel 400 including vulnerable plaque 430.

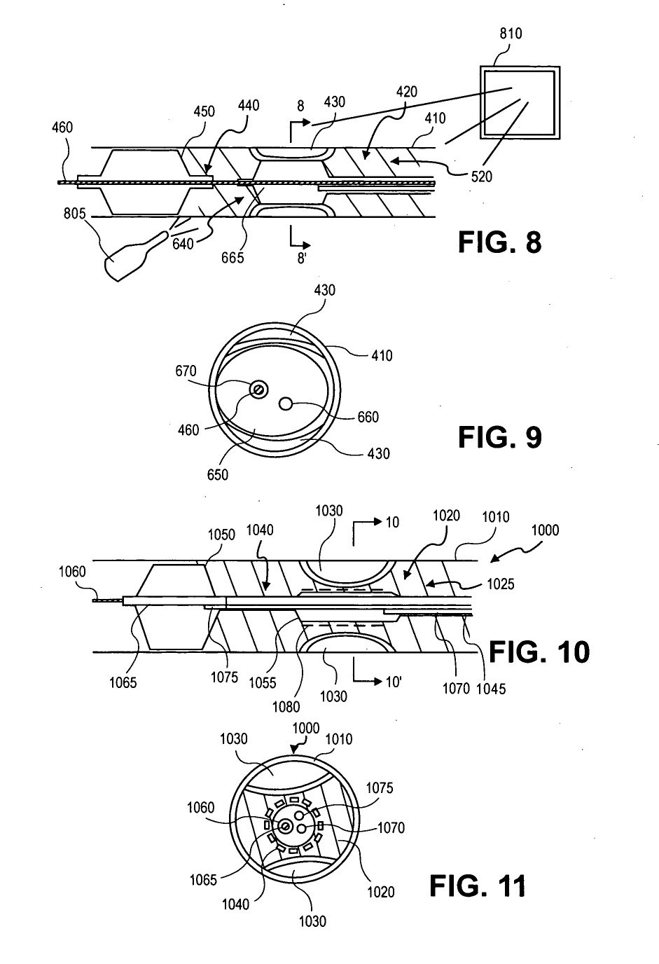

FIG. 8 shows blood vessel 410 following the inflation of balloon 650. In one embodiment, balloon 650 is inflated to a point where contrast agent 520 can no longer be detected over vulnerable plaque 430. In the embodiment where angiographic or fluoroscopic techniques are to be utilized to assess the circularity of lumen 420 of blood vessel 400, balloon 650 may be inflated with a non-radiopaque material such as saline. Contrast agent 520 substantially or totally disappears essentially when balloon 650 circumferentially touches the blood vessel (including the fibrous cap of vulnerable plaque 430) and displaces the contrast agent. In one embodiment, angiographic techniques may be used to assess displacement of contrast agent 520. Representatively, contrast agent 520 may be a radiopaque solution that may be detected using x-ray. FIG. 8 shows x-ray source 805 transmitting x-rays on blood vessel 410.

FIG. 9 shows a cross-sectional side view of blood vessel 410 to line 8-8′ of FIG. 8. FIG. 9 shows balloon 650 inflated to circumferentially contact an inner wall of blood vessel 410 and to contact vulnerable plaque 430. FIG. 9 shows that balloon 650 modifies a lumen of blood vessel 410 from a non-circular cross-sectional shape as shown in FIG. 7 to a shape generally approaching that of a circle. The contracting of vulnerable plaque 430 by balloon 650, in one embodiment, is sufficient to re-shape lumen 420 of blood vessel 400 without rupturing the fibrous cap of vulnerable plaque 430. It is believed that the contact may produce some injury to wall 410 of blood vessel 400 and to vulnerable plaque 430. This injury will tend to induce tissue growth which will strengthen the fibrous cap.

In the embodiment described above with reference to FIGS. 4-9 and the accompanying text, a balloon is used to modify the shape of a lumen at a location of the blood vessel including a vulnerable plaque. Following the modification, the balloon may be removed, for example, by deflating the balloon to a minimal profile and retracting the balloon. The downstream balloon (balloon 450) may be removed in a similar manner.

In one embodiment, the contacting of a vulnerable plaque by a balloon in the context of reshaping the lumen is sufficient to modify the shape of the blood vessel and reduce the stress on the vulnerable plaque following the removal of the balloon. In another embodiment, there may be a desire to support the vulnerable plaque or to assist in the maintenance of the shape of the lumen by implanting a structural device such as a stent in the blood vessel. Thus, in another embodiment, a stent may be placed over balloon 650 and deployed over vulnerable plaque 430 with the expansion of balloon 450. Care must be taken when deploying a stent not to rupture fibrous cap of vulnerable plaque 430. In this regard, one target of this embodiment, and stent deployments described herein, is apposition or putting the stent in contact with the vulnerable plaque with minimum force applied to the vulnerable plaque by the stent. Representatively, a stent may be anchored to a blood vessel wall with a relatively small force possibly applied in a region not including the vulnerable plaque or a stent may be configured to have a varied lumen diameter so at a region of interest or treatment site including a vulnerable plaque, the stent outside diameter is less than an outside diameter of the stent at a location not including the vulnerable plaque. Examples of stents having varied diameters are presented below.

FIGS. 10-13 show another embodiment of a device and technique for modifying a shape of a blood vessel at a location including a lesion or vulnerable plaque. FIG. 10 shows blood vessel 1010 including lumen 1020 therethrough. Blood vessel 1010 includes vulnerable plaque 1030 located within the blood vessel and tending to modify a cross-sectional shape of lumen 1020 (e.g., modify to a non-circular or oblong cross-section).

Disposed within blood vessel 1000 is catheter assembly 1040. FIG. 10 shows only a distal portion of catheter assembly 1040. Catheter assembly 1040 has a tandem balloon configuration including distal balloon 1050 and proximal balloon 1055 aligned in series at a distal portion of the catheter assembly. Catheter assembly 1040 also includes primary cannula 1045 having a length that extends from a proximal end of catheter assembly 1040 (e.g., located external to a patient during a procedure) to connect with a proximal end or skirt of proximal balloon 1055. Primary cannula 1045 has a lumen therethrough that includes inflation cannula 1070 and inflation cannula 1075. Inflation cannula 1070 extends from a proximal end of catheter assembly 1040 to a point within balloon 1055. Inflation cannula has a lumen therethrough allowing balloon 1055 to be inflated through inflation cannula 1070. In this embodiment, balloon 1050 is inflated through a separate inflation lumen. Inflation cannula 1075 has a lumen therethrough allowing fluid to be introduced into balloon 1050 to inflate the balloon. In this manner, balloon 1050 and balloon 1055 may be separately inflated. Each of inflation cannula 1070 and inflation cannula 1075 extend from, in one embodiment, a proximal end of catheter assembly 1040 to a point within balloon 1050 and balloon 1055, respectively.

Catheter assembly 1040 also includes guidewire cannula 1065 extending, in this embodiment, through each of balloon 1050 and balloon 1055 to a distal end of catheter assembly 1040. Guidewire cannula 1065 has a lumen therethrough sized to accommodate guidewire 1060. Catheter assembly 1040 may be an over the wire (OTW) configuration where guidewire cannula extends from a proximal end (external to a patient during a procedure) to a distal end of catheter assembly 1040. In another embodiment, catheter assembly 1040 is a rapid exchange (RX) type catheter assembly and only a portion of catheter assembly 1040 (a distal portion including balloon 1050 and balloon 1055) is advanced over guidewire 1060. In a rapid exchange catheter assembly, typically the guidewire cannula/lumen extends from the distal end of the catheter to a proximal guidewire port space distally from the proximal end of the catheter assembly. The proximal guidewire port is typically spaced a substantial distance from the proximal end of the catheter assembly.

In the embodiment shown in FIG. 10, catheter assembly 1040 includes a deployable stent. FIG. 10 shows stent 1080 positioned on balloon 1050. In one embodiment, stent 1080 has a length dimension as long as a length dimension of a working length of balloon 1055. Typically, a balloon such as balloon 1055 includes a proximal skirt connected to primary cannula 1045, a medial working length, and a distal skirt connected to distal extending guidewire cannula 1065. In one embodiment, a length of a working length of balloon 1055 is longer than a length dimension of vulnerable plaque 1030. In this manner, stent 1080, which has a length similar to a length of the working length of balloon 1055 is longer than vulnerable plaque 1030. In this manner, stent 1080 may be anchored to the blood vessel possibly without anchoring to vulnerable plaque 1030.

FIG. 10 shows an embodiment of a procedure where balloon 1050 located downstream of vulnerable plaque 1030. Balloon 1050 is shown in an expanded or inflated state to occlude lumen 1020. At this point, balloon 1055, on the other hand, is not expanded or inflated or is only partially expanded or inflated so as not to contact vulnerable plaque 1030 or occlude lumen 1020. FIG. 10 also shows contrast agent 1025 introduced into lumen 1020. Contrast agent 1025 tends to pool around balloon 1055 and vulnerable plaque 1030 due to the downstream occlusion of the vessel caused by balloon 1050.

FIG. 11 shows a cross-sectional view through line 10-10′ of FIG. 10. FIG. 11 shows a shape of lumen 1020 of blood vessel 1000 having an irregular shape (e.g., an oblong or non-circular shape). Balloon 1055 of catheter assembly 1040 is shown within lumen 1020 and is shown in a non-expanded or non-inflated state so as not to occlude the lumen. FIG. 11 shows contrast agent 1025 disposed around balloon 1055 and vulnerable plaque 1030. Stent 1080 is shown on balloon 1055.

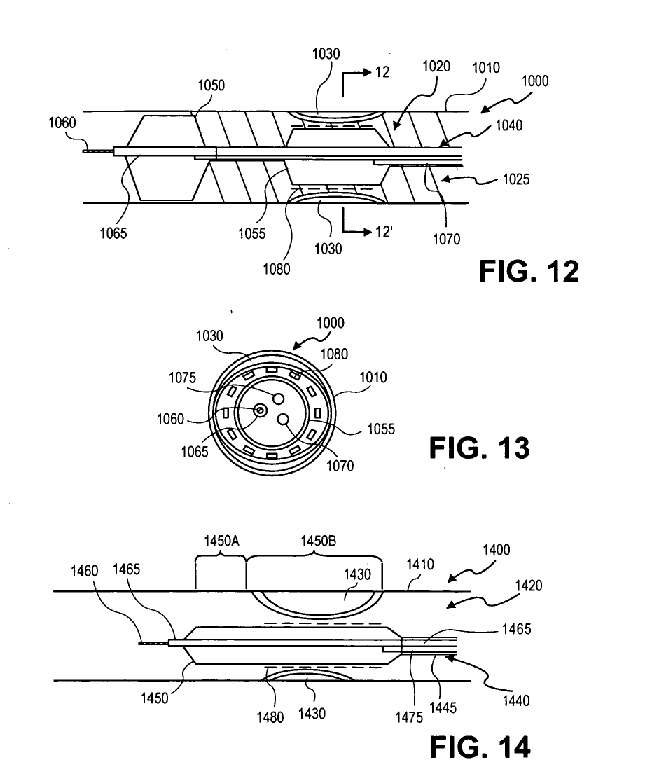

FIG. 12 shows blood vessel 1000 following the expansion or inflation of balloon 1055. In one embodiment, balloon 1055 is expanded using a non-radiopaque solution such as saline. Contrast agent 1025, on the other hand, may be a radiopaque material that may be detected through angiographic or fluoroscopic techniques. In one embodiment, balloon 1055 is expanded until the presence of contrast agent 1025 over vulnerable plaque 1030 substantially disappears, essentially when balloon 1055 and stent 1080 circumferentially touch wall 1010 and vulnerable plaque 1030 and displace contrast agent 1025.

FIG. 13 shows a cross-sectional side view through line 12-12′ of FIG. 12. FIG. 13 shows balloon 1055 expanded to a point where balloon 1055 and stent 1080 (particularly, stent 1080) contact wall 1010 and vulnerable plaque 1030. FIG. 13 illustrates that, in response to the expansion of balloon 1050, a lumen of blood vessel 1000 is modified into a shape approaching that of a circle as compared to the oblong shape shown in FIG. 11.

Following expansion of balloon 1055, balloon 1055 may be deflated to minimize its profile and balloon 1050 may be similarly deflated. Catheter assembly 1040 may then be removed from the blood vessel leaving stent 1080 in an area of blood vessel including vulnerable plaque 1030. In one embodiment, stent 1080 may be anchored to wall 1010, blood vessel 1000 on either or both of the proximal and distal side of vulnerable plaque 1030. Stent 1080 may provide some structural support to vulnerable plaque 1030 to inhibit its rupture.

FIGS. 14-17 show another embodiment of a catheter assembly in a blood vessel including a vulnerable plaque. FIG. 14 shows blood vessel 1400 including vessel wall 1410 and lumen 1420. Disposed within blood vessel 1400 is vulnerable plaque 1430. A build-up of vulnerable plaque 1430 tends to modify a lateral cross-sectional shape of lumen 1420 from circular to irregular (e.g., oblong or non-circular).

FIG. 14 also shows a distal portion of catheter assembly 1440. In this view, catheter assembly 1440 includes primary cannula 1445 having a lumen therethrough. Disposed within a lumen of primary cannula 1445 is guidewire cannula 1465 and inflation lumen 1475. Connected to a distal end of primary cannula 1445 is balloon 1450. In one embodiment, balloon 1450 has a working length that extends the length of a lesion of vulnerable plaque and an additional length. Thus, as illustrated in FIG. 14, in one embodiment for placing catheter assembly 1440 at a region of interest or treatment site, catheter assembly 1450 is percutaneously advanced from a femoral or radial artery to a coronary artery with portion 1450A located in the blood vessel at a location downstream from vulnerable plaque 1430, and portion 1450B located in the blood vessel at the same location as vulnerable plaque 1430. FIG. 14 shows the region of interest in blood vessel 1400 including catheter assembly 1440. Imaging techniques such as OCT and IVUS may be used to identify the location in a blood vessel and position the catheter assembly. At least a distal portion of catheter assembly 1440 may be advanced over guidewire 1460 (over guidewire cannula 1465) to the region of interest.

As shown in FIG. 14, a distal portion of catheter assembly 1440 includes primary cannula 1445 containing guidewire cannula 1465 and inflation cannula 1475.

In the embodiment shown, a working length of balloon 1450 may have similar expansion characteristics throughout its length. To modify the expansion characteristics, stent 1480 is placed over a portion of balloon 1450. As shown in FIG. 14, stent 1480 is placed over balloon 1450 at portion 1450B while portion 1450A is free. Accordingly, introducing a fluid through a lumen of inflation cannula 1475 will tend to cause portion 1450A to expand more rapidly than portion 1450B.

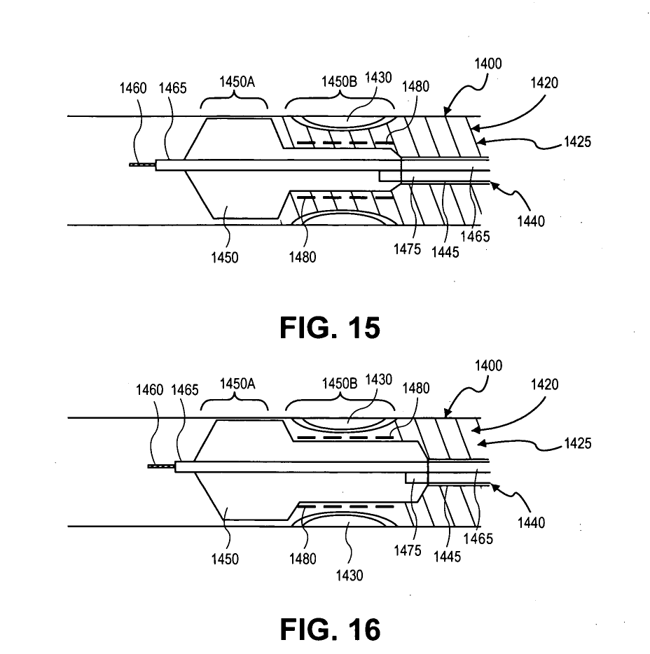

FIG. 15 shows blood vessel 1400 following the partial expansion of balloon 1450. As illustrated, portion 1450A of balloon 1450 expands more rapidly than portion 1450B. In one embodiment, portion 1450A expands to a diameter substantially equivalent to an interior diameter of blood vessel 1400 so that portion 1450A occludes lumen 1420 of the blood vessel. At this point, portion 1450B has a diameter less than a diameter of blood vessel 1400 modified by vulnerable plaque 1430. Contrast agent 1425 may be introduced into lumen 1420 of blood vessel 1400 and pool at and around vulnerable plaque 1430.

FIG. 16 shows blood vessel 1400 following the further expansion of balloon 1450. According to one embodiment, balloon 1450 is expanded (inflated) until portion 1450B circumferentially touches vulnerable plaque 1430 and vessel wall 1410 and displaces contrast agent 1425. As noted above, this may be visualized through angiographic or fluoroscopic techniques using a radiopaque material as a contrast agent and a non-radiopaque material to inflate balloon 1450.

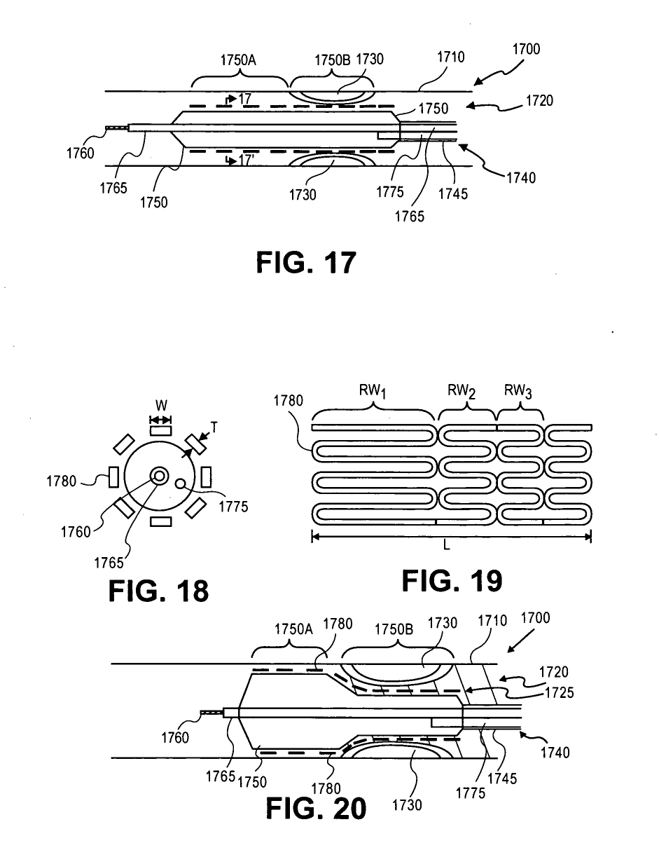

FIG. 17 shows a blood vessel having a catheter assembly disposed in a lumen thereof. Referring to FIG. 17, blood vessel 1700 includes blood vessel wall 1710 defining lumen 1720. Disposed within blood vessel 1700 is vulnerable plaque 1730. Vulnerable plaque 1730 tends to modify a cross-sectional shape of lumen 1720 from generally circular to irregular or oblong.

FIG. 17 shows catheter assembly 1740 disposed within lumen 1720 defined by blood vessel wall 1710. FIG. 17 shows a distal portion of catheter assembly 1740. Catheter assembly 1740 includes primary cannula 1745 having a lumen therethrough, the lumen sized to contain at least guidewire cannula 1765 and inflation cannula 1775. Each of guidewire cannula 1765 and inflation cannula 1775 has a lumen therethrough. A lumen of guidewire cannula 1765 is of a size to include guidewire 1760. Catheter assembly 1740 also includes balloon 1750 connected at a proximal and to primary cannula 1745 and a distal end to guidewire cannula 1765. A distal end of inflation cannula 1775 is disposed within balloon 1750.

In the embodiment shown in FIG. 17, a working length of balloon 1750 is longer than a length dimension of vulnerable plaque 1730. Thus, as shown in FIG. 17, balloon 1750 of catheter assembly 1730 is positioned, in one embodiment, such that a portion of the balloon extends beyond (downstream from) a length of vulnerable plaque 1730. FIG. 17 shows portion 1750A in lumen 1720 extending in a distal direction beyond a location of vulnerable plaque 1730. Portion 1750B is positioned at a location in lumen 1720 of blood vessel 1700 of vulnerable plaque 1730.

In one embodiment, the working length of balloon 1750 has similar expansion characteristics across its length. Overlying the working length of balloon 1750 is stent 1780. In this embodiment, the expansion characteristics of stent 1780 are varied across its length. In one embodiment, the expansion characteristics of stent 1780 are modified such that, relative to balloon 1750 and its placement in blood vessel 1700, a distal portion of stent 1780 expands more readily than a proximal portion. Thus, relative to balloon 1750, that portion of stent 1780 overlying portion 1750A expands more easily than that portion of stent 1780 overlying portion 1750B.

There are various ways to modify the expansion characteristics of a stent. A stent typically includes a plurality of radially expandable cylindrical elements (a plurality of struts) disposed generally coaxially in rings. The rings may be interconnected by connecting elements (a plurality of links). FIG. 18 shows a cross-sectional side view of catheter assembly 1740 at line 17-17′ of FIG. 17. FIG. 18 illustrates stent 1780 having struts with a width, W and thickness, T. A representative strut width, W, for a typical stent is on the order of 0.0025 inches to 0.0035 inches. A representative thickness, T for a typical stent is on the order of 0.002 inches to 0.010 inches. By increasing either or both of a stent thickness, T or width, W, stent 1780 becomes harder to expand. Thus, in one embodiment, the thickness, T and width, W of struts overlying portion 1750B of balloon 1750 are increased relative to struts overlying portions 1750A. One example is increasing the thickness, T, and/or width, W, of struts overlying portion 1750B by 30 percent.

In addition to modifying the strut width or strut thickness, a ring width of a strut (a ring of struts) may be modified to modify the expansion characteristics of stent 1780. FIG. 19 shows a flattened portion of stent 1780 according to another embodiment. In this embodiment, a ring width, RW, is modified along a length of stent 1780 to modify its expansion characteristics. In general, increasing the ring width, RW, of a stent tends to make the stent expand more easily. Thus, FIG. 19 shows a first portion of strut 1780 having a ring width, RW₁, that is greater than a second portion, RW₂, and a third portion, RW₃. The longer ring width strut, portion with RW₁, in one embodiment, would be positioned over portions 1750A of balloon 1750. The second portion, RW₂, has a ring width equal to or less than a first portion, RW₁, and greater than a third portion, RW₃, and therefore might be located in a transition between portion 1750A and portion 1750B of balloon 1750. The smaller ring width portion, portion with RW₃, would be located over portion 1750B of balloon 1750. In one embodiment, first portion, RW₁ and second portion, RW₂ are similar and are fifty percent greater than third portion, RW₃ (e.g., RW₁=1.5 mm and RW₃=1.0 mm).

FIG. 20 shows blood vessel 1700 following the partial expansion of balloon 1750 of catheter assembly 1740. As illustrated, portion 1750A of balloon 1750 is expanded to a greater diameter than portion 1750B at this point. The greater expansion of portion 1750A is due to the modification of the expansion characteristics of stent 1780. As illustrated, portion 1750A is expanded to an amount sufficient to substantially or totally occlude lumen 1720 of blood vessel 1700. Following the partial expansion of balloon 1750, a contrast agent is introduced into the blood vessel. Contrast agent 1725 tends to pool around balloon portion 1750B and vulnerable plaque 1730.

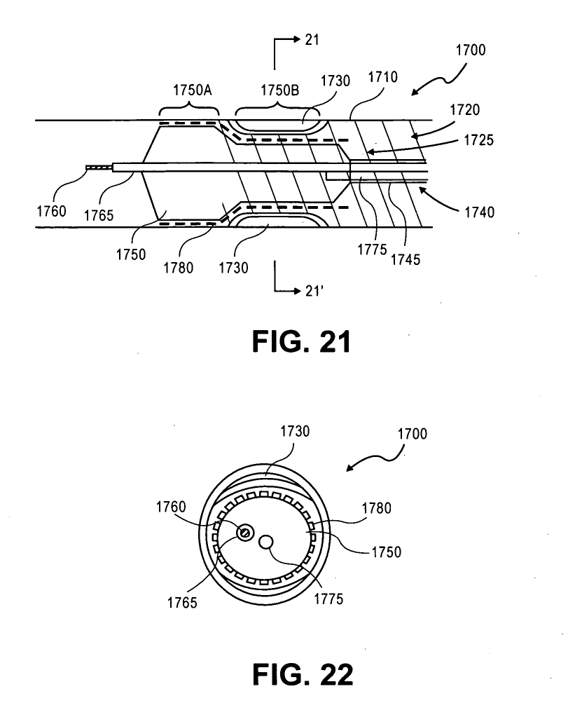

FIG. 21 shows blood vessel 1700 following the further expansion of balloon 1750. The further expansion includes the expansion of portion 1750B. In one embodiment, balloon 1750 is expanded to a point that stent 1780 and possibly a wall of balloon 1750 circumferentially touches wall 1710 of blood vessel 1700 and vulnerable plaque 1730 and displaces contrast agent 1725. Such expansion may be visualized by selecting contrast agent 1725 that is a radiopaque material and a fluid to expand balloon 1750 that is non-radiopaque.

FIG. 21 shows blood vessel 1700 following the further expansion of balloon 1700. Using fluoroscopic techniques, balloon 1750 can be expanded using a non-radiopaque fluid, to a point at which the contrast material over balloon portion 1750B is minimized or disappears. The contrast agent is minimized or disappears essentially when stent 1780 or balloon 1750 circumferentially touches the blood vessel wall and displaces the contrast agent. FIG. 22 shows a cross-sectional side view through line 21-21′ of FIG. 21. FIG. 22 shows the blood vessel having lumen 1720 that is essentially circular and modified from an oblong or non-circular condition caused by vulnerable plaque 1730.

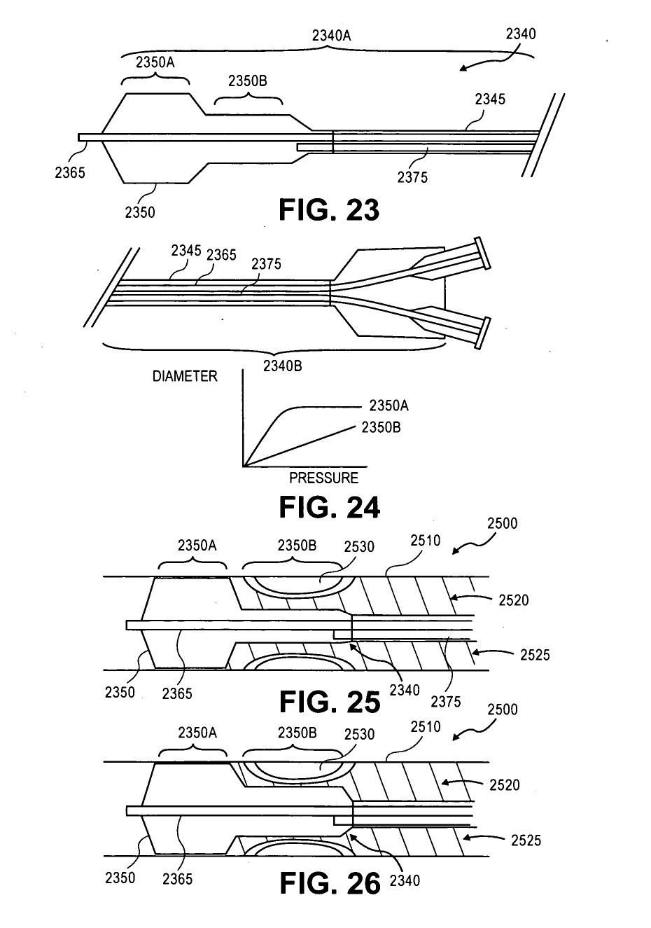

FIG. 23 shows an embodiment of a catheter assembly. Referring to FIG. 23, catheter assembly 2340 includes distal portion 2340A intended for insertion into a body lumen, such as a blood vessel, and proximal portion 2340B intended to remain external to a patient when catheter assembly 2340 is in use. Catheter assembly 2340 includes primary cannula or tubular member 2345 extending from proximal portions 2340B through distal portion 2340A. In one embodiment, primary cannula 2345 has a length such that catheter assembly 2340 may be percutaneously inserted into either a femoral or a radial artery and advanced to a coronary artery (e.g., left coronary artery, left anterior descending artery, right coronary artery, etc.). In one embodiment, primary cannula 2345 has a lumen that is sized to contain at least two cannulas or tubular members (e.g., a two-lumen shaft). As illustrated, primary cannula 2345 includes guidewire cannula 2365 and inflation cannula 2375. In one embodiment, catheter assembly 2340 is an over-the-wire (OTW) catheter assembly where guidewire cannula 2365 extends from a proximal end of the catheter assembly to a distal end. In another embodiment (not shown), catheter assembly 2340 is a rapid exchange (RX) type catheter assembly where guidewire catheter 2365 extends through only a portion of primary cannula 2345 (e.g., a distal portion).

FIG. 23 shows balloon 2350 connected to primary cannula 2345. Balloon 2350 is illustrated in an inflated state. Balloon 2350 may be inflated through inflation cannula 2375. Inflation cannula 2375 extends through primary cannula 2345 from proximal portion 2340B and distally terminates within balloon 2350.

As illustrated in FIG. 23, balloon 2350 has two different inflation diameters. Balloon 2350 includes portion 2350A that has a greater inflation diameter than portion 2350B. In one embodiment, portion 2350A has an inflation diameter equivalent to a diameter of a blood vessel (e.g., coronary artery). A representative diameter is on the order of approximately two millimeters (mm) to 5 mm. Portion 2350B has an inflation diameter less than a diameter of portion 2350A. A typical vulnerable plaque modifies the interior diameter of a blood vessel by about 0.3 mm to 1.0 mm. In one embodiment, an inflated diameter of portion 2350B will be sufficient to contact a vulnerable plaque within a blood vessel without stretching the vulnerable plaque. Accordingly, an exterior diameter of portion 2350B will be 0.3 mm to 1.0 mm less than an inflated diameter of portion 2350A.

In one embodiment, portion 2350A of balloon 2350 is non-compliant. In other words, portion 2350A may expand to a particular diameter and increasing the inflation pressure will not increase the diameter of the balloon. At the same time, portion 2350B may be compliant, meaning that increasing pressure will increase the diameter of portion 2350B beyond, for example, a pressure necessary to fully inflate portion 2350A. FIG. 24 shows a representation of the expansion pressure of portion 2350A and 2350B. As illustrated in FIG. 24, portion 2350A will expand to a predetermined diameter at a given inflation pressure, and once that pressure is reached, portion 2350A will not expand the predetermined diameter. At the same time, portion 2350B will expand, albeit not as great, with an increase in inflation pressure without reaching a limit within the inflation pressure necessary to fully inflate balloon 2350.

In one embodiment a suitable material for balloon 2350A is expanded polytetrafluoroethylene (ePTFE). To form portion 2350B that is non-compliant, ePTFE ribbon may be wound around a mandrel having a size that is slightly larger (e.g., 1-2 mm larger) than a desired diameter of portion 2350A when inflated. To make portion 2350A non-compliant, multiple layers of ePTFE windings may be employed. Following windings and multiple layers, the ePTFE material may be fused to form portion 2350B. To form compliant portion 2350B, ePTFE material may also be used. In one example, the number of layers of ePTFE windings is less than the number of layers of windings selected for non-compliant portion 2350A. In one embodiment, compliant portion 2350A is formed on a mandrel having a diameter that is less than a diameter selected for portion 2350A and is sized to target a diameter of a blood vessel including a vulnerable plaque.

As noted above, in one embodiment, portion 2350A is non-compliant. Portion 2350A may be a material that achieves its target diameter at a pressure of less than about one to four atmospheres, to inflate balloon 2350, and inflation fluid may be introduced through a lumen of inflation cannula 2375. Portion 2350A will reach its target diameter at a pressure of less than one to four atmospheres while portion 2350B may continue to expand at pressures greater than one to four atmospheres. Although ePTFE is described as a suitable balloon material, other materials such as PEBAX, Nylon or polyurethane are suitable for forming a balloon with variable diameter.

FIG. 25 shows a cross-sectional side view of a blood vessel having catheter assembly 2340 disposed therein. Blood vessel 2500 includes vessel wall 2510 having lumen 2520 therethrough. FIG. 25 shows vulnerable plaque 2530 formed in blood vessel 2500 and modifying a lateral cross-sectional shape of lumen 2520.

FIG. 25 shows distal portion 2340A of catheter assembly within blood vessel 2500. In one embodiment, catheter assembly 2340 may be placed at a region of interest or treatment site within blood vessel 2500 by advancing at least a portion of catheter assembly 2340 over a guidewire using guidewire cannula 2365. A guidewire is not shown in the figure. In one embodiment, catheter assembly 2340 is advanced to a point in the blood vessel where portion 2350A of balloon 2350 is downstream from vulnerable plaque 2530. Portion 2350B is positioned at a location in blood vessel 2500 including vulnerable plaque 2530. FIG. 25 shows catheter assembly 2340 following the expansion of portion 2350A of balloon 2350 to a diameter sufficient to occlude lumen 2520 of blood vessel 2500. FIG. 25 also shows contrast agent 2525 introduced into blood vessel 2500. Contrast agent 2525 tends to pool around vulnerable plaque 2530 and portion 2350B of balloon 2350. At this point, portion 2350B is not inflated to a target diameter so that portion 2350B in not in contact with vulnerable plaque 2530 or vessel 2510.

FIG. 26 shows a cross-sectional side view of blood vessel 2500 following the expansion of portion 2350B of balloon 2350. In one embodiment, portion 2350B is expanded until minimal or no contrast agent 2525 can be detected around portion 2350B or vulnerable plaque 2530. Angiographic or fluoroscopic techniques as described above may be used to detect a desired expansion of portion 2350B.

As described above, balloon 2350 of catheter assembly 2340 is used to modify a diameter of lumen 2520 of blood vessel 2500. In one embodiment, a buildup of vulnerable plaque 2530 modifies the shape of lumen 2520 from circular to an irregular or oblong shape. Expansion of portion 2350B tends to establish a circular lateral cross-section. Following modification, balloon 2350 may be deflated to a minimum profile and catheter assembly 2340 removed. In another embodiment, a stent may be placed on portion 2350B and deployed in the blood vessel to provide structural support to vulnerable plaque 2530.

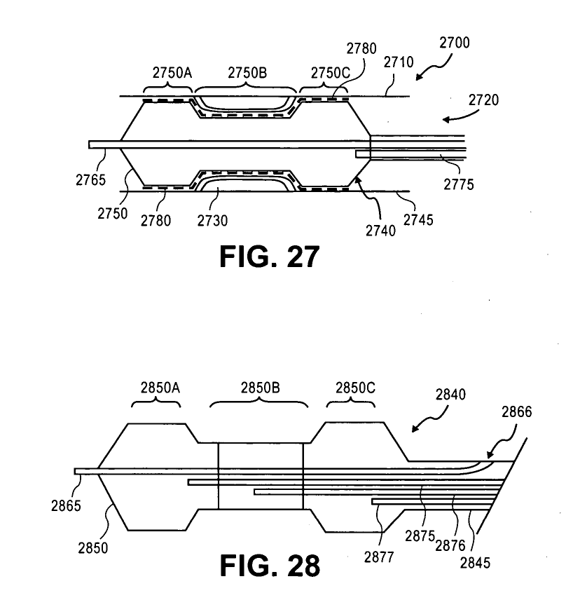

FIG. 27 shows a cross-sectional side view of a blood vessel. Blood vessel 2700 includes vessel wall 2710 having lumen 2720 therethrough. Blood vessel 2700 also includes lesion or vulnerable plaque 2730 disposed in a portion of the blood vessel and modifying a lateral cross-sectional diameter of lumen 2720 from a generally circular shape to an irregular or oblong shape.

FIG. 27 shows catheter assembly 2740 disposed within blood vessel 2700. Only a distal portion of catheter assembly 2740 is shown. Catheter assembly 2740 includes primary cannula or tubular member 2745 that may extend from a proximal portion external to a patient to a distal portion adjacent a region of interest or treatment site. Primary cannula 2745 has a lumen therethrough that is sized to accommodate at least two cannulas or tubular members (e.g., a two-lumen shaft). FIG. 27 shows guidewire cannula 2765 and inflation cannula 2775 disposed within a lumen of primary cannula 2745. Guidewire cannula 2765 may extend to a proximal end of catheter assembly 2740 (an OTW configuration) or may extend only through a distal portion of the catheter assembly (an RX configuration). In one embodiment, inflation cannula 2775 extends from a proximal end of catheter assembly 2740 beyond a distal end of primary cannula 2745.

Connected at a proximal end to primary cannula 2745 is balloon 2750. As illustrated, a working length of balloon 2750 includes multiple inflation diameters. FIG. 27 shows balloon 2750 in an inflated or expanded state having portion 2750A, portion 2750B, and portion 2750C. Each portion of balloon 2750 is inflated using inflation cannula 2775. Overlying balloon 2750 in each of portion 2750A, portion 2750B, and portion 2750C is stent 2780. FIG. 27 shows portion 2750A of balloon 2750 positioned downstream (distal) to vulnerable plaque 2730. Portion 2750C of balloon 2750 is positioned upstream (proximal) to vulnerable plaque 1730. Portion 2750B of balloon 2750 is positioned in blood vessel 2700 at a location including vulnerable plaque 2730. Overlying a working length of balloon 2750 in each of portion 2750A, portion 2750B and portion 2750C is stent 2780.

As shown in FIG. 27, portion 2750A and portion 2750C are expanded to a diameter sufficient to substantially or totally occlude blood vessel 2700. In one embodiment, portion 2750A and portion 2750C are expanded to a diameter sufficient to bring stent 2780 into contact with blood vessel wall 2710 of blood vessel 2700. Thus, portion 2750A and portion 2750C serve, in one aspect, to anchor stent 2780 in place. Accordingly, an expanded diameter of portion 2750A and portion 2750B is guided by a diameter of lumen 2720 of blood vessel 2700. In one embodiment, portion 2750A and portion 2750C are selected so that they have an expanded diameter equivalent to a diameter of blood vessel 2720. A reference diameter is on the order of two millimeters to five millimeters.

Unlike portion 2750A and portion 2750C, portion 2750B of balloon 2750 is selected to have an expanded diameter sufficient to reshape or to modify a shape of blood vessel 1720 at a location including vulnerable plaque 2730. The expanded diameter should be sufficient to modify the shape of the blood vessel without rupturing the vulnerable plaque. the expanded diameter should also account for the presence of the stent 2780 with an objective to use the stent as support or scaffolding for vulnerable plaque 2730 or neointimal tissue growth. Accordingly, in one embodiment, an expanded diameter of portion 2750B is selected such that stent 2780 is in contact with vulnerable plaque 2730. A typical vulnerable plaque may modify the inner diameter of a blood vessel by 0.3 mm to 1.0 mm. Accordingly, in one embodiment, portion 2750B has an expanded diameter approximately 0.3 mm to 1.0 mm less than portion 2750A or portion 2750C. The diameters of portion 2750A, portion 2750B and portion 2750C may be preselected and molded to a chosen size based on the referenced diameters of a blood vessel and the stenosis severity of the vulnerable plaque.

Another technique for varying a diameter of balloon 2750 is to make portion 2750A and portion 2750C non-compliant while portion 2750B is compliant. In one embodiment, portion 2750A and portion 2750C are selected to be inflated to a predetermined standard diameter of relatively low inflation pressure, for example, under four atmospheres, while portion 2750B requires greater inflation pressure for expansion (e.g., greater than four atmospheres). In operation, portion 2750A and portion 2750C would be inflated to an expanded diameter initially and portion 2750B would then be inflated to a desired expanded diameter by increasing the inflation pressure beyond the pressure necessary to inflate portion 2750A or portion 2750C. Since portion 2750A and portion 2750C are non-compliant, the increase in inflation pressure would have minimal effect on expanded diameter of portion 2750A or portion 2750C.

FIG. 28 shows another embodiment of a catheter assembly including a balloon having multiple different inflated diameters. FIG. 28 shows only a distal portion of the catheter assembly. Referring to FIG. 28, catheter assembly 2840 includes primary cannula or tubular member 2845 that has a length suitable such that catheter assembly 2840 may be percutaneously inserted into either a femoral or radial artery and advanced to a coronary artery. FIG. 28 shows balloon 2850 connected to a distal end of primary cannula 2845. In this embodiment, balloon 2850 includes portion 2850A, portion 2850B and portion 2850C. Balloon 2850 is shown in an expanded state.

In one embodiment, primary cannula 2845 has a lumen that is sized, at least at a distal portion, to include at least four cannulas or tubular members (e.g., a four-lumen shaft). As illustrated, primary cannula 2845 includes guidewire cannula 2865. In this embodiment, catheter assembly 2840 is a rapid exchange (RX) type catheter assembly with guidewire cannula extend through a distal portion of primary cannula 2845 rather than from a proximal end of catheter assembly 2840. FIG. 28 shows guidewire cannula 2865 extending from port 2866 through a distal end of the catheter assembly 2840.

Also contained within primary cannula 2845 are three inflation cannulas. FIG. 28 shows inflation cannula 2875 having a distal end within portion 2850A of balloon 2850; inflation cannula 2876 having a distal end within portion 2850B; and inflation cannula 2877 having a distal end within portion 2850C. Each inflation cannula extends, in one embodiment, from a proximal end of catheter assembly 2840 (intended to be external to a patient during a procedure) to a location within a balloon portion.

As shown in FIG. 28, balloon 2850 of catheter assembly 2840 has multiple inflated or expanded diameters. Similar to the embodiment described in FIG. 27, portion 2850A and portion 2850C have an expanded diameter greater than portion 2850B. In one embodiment, portion 2850A is intended to be placed at a position in a blood vessel downstream or distal to a lesion or vulnerable plaque. Portion 2850C of balloon 2850 is intended, in one embodiment, to be positioned at a position upstream or proximal to a lesion of vulnerable plaque. Portion 2850B is intended to be placed within a blood vessel at a location including a lesion of vulnerable plaque. In one embodiment, a stent may be deployed using catheter assembly 2840. The stent may have a length corresponding to a working length of balloon 2850 (including a length of portion 2850A, portion 2850B and portion 2850C). In this manner, portion 2850A and portion 2850B may have an expanded diameter equivalent to a diameter of a blood vessel and may expand to anchor a stent to a blood vessel wall at locations not including a vulnerable plaque.

By having separately controlled portions of a balloon, the particular expanded diameters may be controlled. In addition, the separate inflation lumens allow the angiographic or fluoroscopic technique described earlier to be employed. For example, balloon portion 2850A may be expanded to occlude a blood vessel, followed by introduction of a radiopaque contrast agent. Portion 2850B could then be expanded to a desired diameter (to a diameter where the contrast agent is no longer detectable). Finally, portion 2850C could be expanded to, for example, deploy a stent. In another embodiment, portion 2850A and portion 2850C may be filled using a single cannula while portion 2850B is inflated using a separate cannula (inflation lumen). Such a configuration would reduce the profile of catheter assembly 2840 by allowing the reduction of primary cannula 2845 compared with the embodiment shown in FIG. 28.

Embodiments of catheter assembly are described with respect to FIG. 27 and FIG. 28 that may be employed in a blood vessel including a vulnerable plaque to reshape a lumen of the blood vessel and/or possibly to support the vulnerable plaque (e.g., inhibit rupture). As noted, stents may be deployed as part of this effort. Thus, a stent may aid in reshaping a lumen of a blood vessel and/or to support a vulnerable plaque (e.g., if the lumen including the vulnerable plaque is close to circular or it is not desired to reshape an irregular lumen). In one embodiment, a stent has a length corresponding to a working length of a balloon having the multiple inflation diameters illustrated in FIG. 27 and FIG. 28. Using balloon 2750 (FIG. 27) as an example, stent 2780 may have a length that extends an entire working length of balloon 2750, including a length equivalent to portion 2750A, portion 2750B and portion 2750C. In another embodiment, a stent may have a length greater than a length of portion 2750B such that it extends at least a portion of the length of portion 2750A and portion 2750C but less than an entire working length of portion 2750A and portion 2750C (e.g., overlaps a portion of each of portion 2750A and portion 2750C). In either embodiment, a stent, such as stent 2780, may have a constant expansion characteristic along its length. A suitable stent is the VISION™ stent design manufactured by Guidant Corporation of St. Paul, Minn. Alternatively, since a vulnerable plaque does not require a stent to have radial strength, a stent similar to a VISION™ stent with narrower and thinner struts could also be used. For example, a VISION™ stent has struts having a strut width of 0.0030 inches and a thickness of 0.0032 inches.

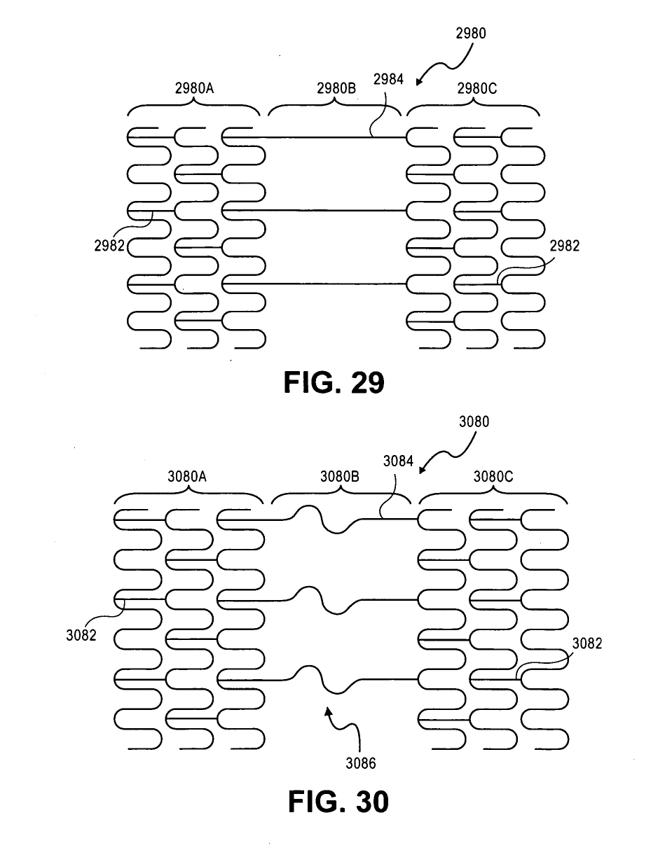

In another embodiment, a stent can be made such that its anchoring portion differs from a portion intended to be positioned in a blood vessel at a vulnerable plaque. FIGS. 29-38 show examples of suitable stent patterns. FIG. 29 shows stent 2980 including portion 2980A, portion 2980B and portion 2980C. Portion 2980A and portion 2980C are intended to be positioned adjacent to a vulnerable plaque and to aid in the anchoring of stent 2980 to a blood vessel. Portion 2980B is intended to be placed at a location in the blood vessel including a vulnerable plaque. Thus, using the example of balloon 2750, portion 2980A is intended to be positioned at a location in a blood vessel downstream of a vulnerable plaque, and portion 2980C is intended to be positioned upstream of a vulnerable plaque. Any reference to portions “A”, “B” and “C”, in FIGS. 30-38 will correspond to this identification.

FIG. 29 shows portion 2980A and portion 2980B each having three rings of struts and six struts per ring. The rings in each portion are in phase and are connected by axial links 2982. Since a vulnerable plaque generally does not require a lot of radial strength, stent 2980 may be configured such that portion 2980B has minimal strut density. FIG. 29 shows portion 2980B having no struts per say but suspension elements 2984 connecting a proximal ring of portion 2980A to a distal ring of portion 2980C. Stent 2980 includes three suspension elements 2984 each disposed axially with a linear profile.

FIG. 30 shows another embodiment of a stent. Stent 3080 includes anchor portion 3080A, portion 3080C and portion 3080B between portion 3080A and portion 3080C. In this embodiment, portion 3080B again has minimal strut or suspension element density since it is intended to be positioned in a blood vessel at a location including a vulnerable plaque. FIG. 30 shows portion 3080A and portion 3080C each having three rings of six struts per ring. The rings of each portion are in phase and are connected by links 3082. Portion 3080B includes three suspension elements 3084 connecting rings of portion 3080A with the rings of portion 3080C. Each suspension element 3084 includes undulation 3086. The undulations in suspension elements 3084 provide the suspension elements with a modifiable strain force allowing, for example, suspension elements 3084 to be stretched.

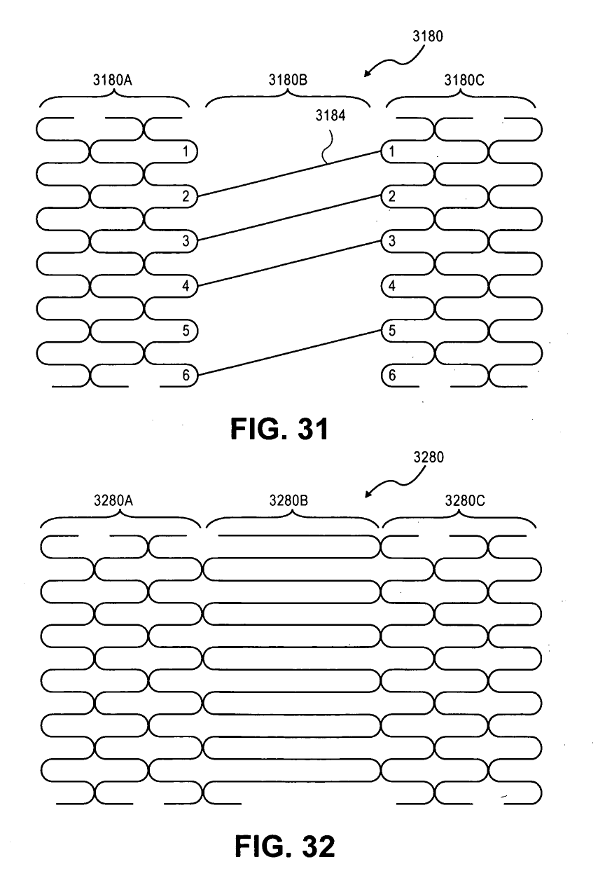

FIG. 31 shows another embodiment of a stent. Stent 3180 includes anchor portion 3180A, portion 3180C and portion 3180B between portion 3180A and portion 3180C. Each of portion 3180A and portion 3180C include three rings of six struts per ring. Adjacent rings are 180 degrees out of phase so that the rings are connected between the crowns and valleys of each strut. Portion 3180B includes four suspension elements 3184 that are connected between crowns and valleys, respectively, of the struts that make up the proximal ring of distal portion 3180A and the distal ring of proximal portion 3180C. As shown, the proximal ring of distal portion 3180A is 180 degrees out of phase with the proximal ring of portion 3180B creating a mirror image one of the other. Using the designation that crowns of a strut project to the left of the page across the stent, as shown, suspension elements 3184 are connected between the rings of portion 3180A and portion 3180C in an offset pattern so that a suspension element is connected between a valley of a second strut in a ring of portion 3180A and a crown of a first strut in a ring of portion 3180C; a valley of a third strut in a ring of portion 3180A and a crown of a second strut in a ring of portion 3180C; etc. The connection of suspension elements 3184 appears diagonal. FIG. 31 also shows suspension elements 3184 clustered in one portion of the stent (e.g., a top portion of the flattened stent as viewed). This clustering may be intended to overlie a vulnerable plaque or not. For example, stent suspension elements 3184 may be asymmetric with more on one side than the other. Many vulnerable plaques are also eccentric and asymmetric, so a denser area of suspension elements 3184 could be aligned and placed over a vulnerable plaque.

FIG. 32 shows another embodiment of a stent. Stent 3280 includes portion 3280A, portion 3280C and portion 3280B between portion 3280A and portion 3280C. Portion 3280A and portion 3280C each have three rings of struts and six struts per ring. The rings in each portion are 180 degrees out of phase with an adjacent ring and the rings are connected between the crowns and valleys. The proximal ring of portion 3280A is also 180 degrees out of phase with the distal ring of portion 3280C.

Portion 3280B of stent 3280 is comprised of a ring of six struts. The struts have a ring width larger than the ring width of the rings that make up portion 3280A or 3280C (e.g., three or four times greater). The struts of portion 3280B are connected between the valleys and crowns of portion 3280A and portion 3280B, respectively, so that the distal ring of portion 3280B is 180 degrees out of phase with the proximal ring of portion 3280A and the distal ring of portion 3280C. In this manner, the stent has less radial strength in portion 3280B which enables it to gently support a vulnerable plaque when the stent is deployed in a blood vessel (e.g., support through apposition).

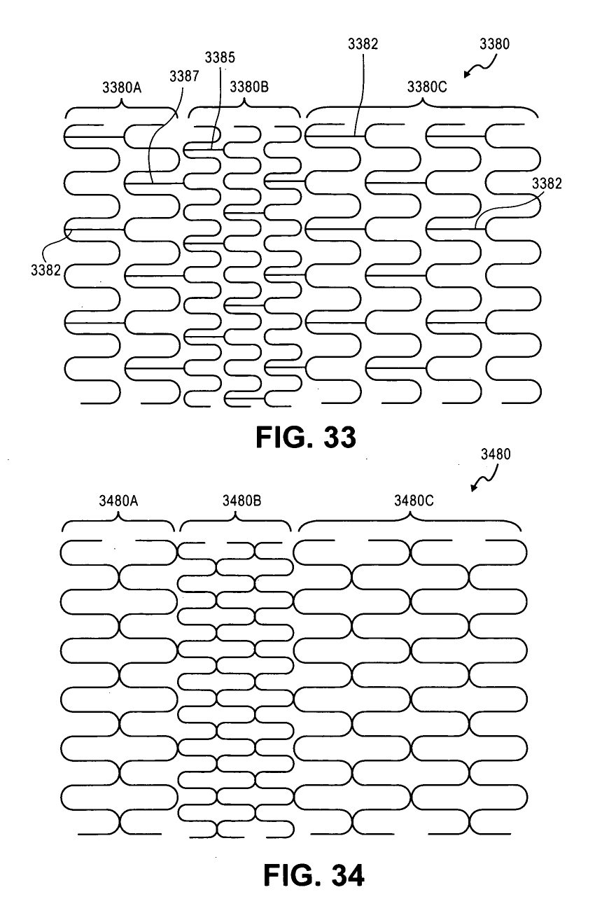

FIG. 33 shows another embodiment of a stent. Stent 3380 includes portion 3380A, portion 3380C and portion 3380B between portion 3380A and portion 3380C. Portion 3380A has two rings of struts and six struts per ring. Portion 3380C has four rings of struts and six struts per ring. Stent 3380 is asymmetric longitudinally with the two rings of portion 3380A and the four rings of portion 3380C. The rings in portion 3380A and portion 3380C are in phase and a crown of every other strut are connected through axial links 3382. The proximal ring of portion 3380A is also in phase with the distal ring of portion 3380C.

Portion 3380B of stent 3380 includes three rings of nine struts. The struts have a ring width that is smaller (e.g., about half size) of the rings that make up portion 3380A or portion 3380C. Each of the rings of portion 3380B are in phase and connected by axial links 3385 at every third strut and the axial links that connect the distal and medial rings are located between different crowns of the axial links that connect the medial and proximal rings. Portion 3380B is connected to portion 3380A and portion 3380C through axial links 3387 between crowns of the individual portions at every other strut relative to portion 3380A or portion 3380B. A stent configured as stent 3380A increases the number of struts in portion 3380B that might overlie a vulnerable plaque.

FIG. 34 shows another embodiment of the stent. Stent 3480 includes portion 3480A, portion 3480C and portion 3480B between portion 3480A and portion 3480C. Similar to stent 3380 in FIG. 33, stent 3480 is asymmetric longitudinally. Portion 3480A has two rings of struts and six struts per ring. Portion 3480C has four rings of struts and six struts per ring. Adjacent rings in portion 3480A are 180 degrees out of phase and the rings are connected between the crowns and valleys. Similarly, adjacent rings in portion 3480C are 180 degrees out of phase and are connected between the crowns and valleys. The proximal rings of portion 3480A is 180 degrees out of phase with the distal ring of portion 3480C.

Portion 3480B of stent 3480 includes three rings of nine struts. Similar to portion 3380B of stent 3380 (see FIG. 33), the struts have a ring width smaller than the ring width of the rings that make up portion 3480A or portion 3480C (e.g., twice as small). The struts of adjacent struts of portion 3480B are 180 degrees out of phase and are connected between the valleys and crowns of the individual rings. Finally, every third crown of the distal ring of portion 3480B is connected to a valley of the proximal ring of portion 3480A. Every third valley of the proximal ring of portion 3480B is connected to a crown of a distal ring of portion 3480C. Similar to stent 3380, portion 3480B is intended to overlie a vulnerable plaque.

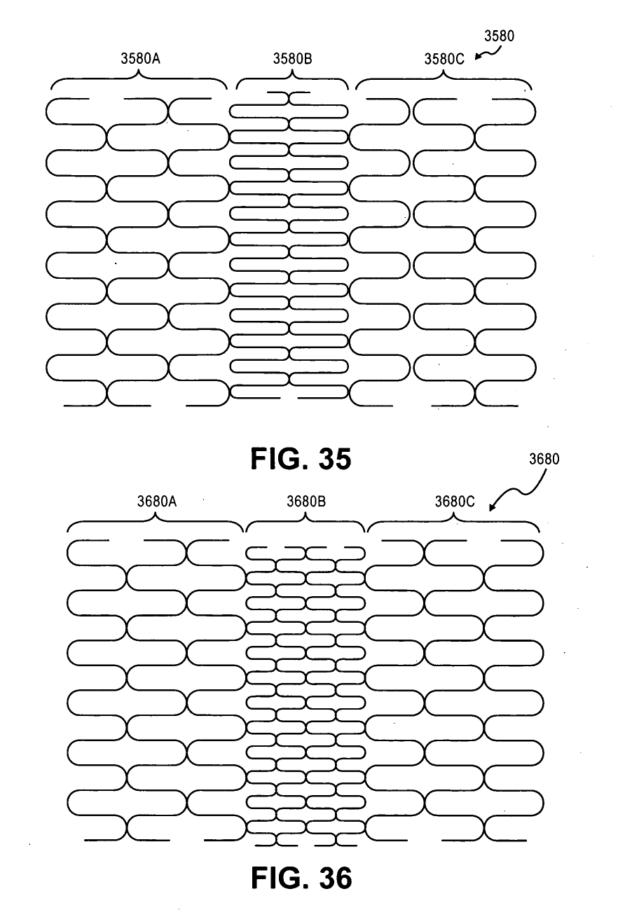

FIG. 35 shows another embodiment of a stent. Stent 3580 includes portion 3580A, portion 3580C and portion 3580B between portion 3580A and portion 3580C. Portion 3580A and portion 3580C each have three rings of struts and six struts per ring. The rings in each portion are 180 degrees out of phase with an adjacent ring and the rings are connected between the crowns and valleys. The proximal ring of portion 3580A is also 180 degrees out of phase with the distal ring of portion 3580C. Unlike stent 3480 (FIG. 34) or stent 3380 (FIG. 33), stent 3580 is symmetric longitudinally.

Portion 3580B of stent 3580 includes two rings of 12 struts. Thus, portion 3580B has more struts (e.g., more crowns and valleys) than portion 3580B and portion 3580C. The struts of each ring are 180 degrees out of phase. A distal ring of portion 3580B is connected at a crown to a valley of the proximal ring of portion 3580A. A proximal ring is connected at a valley to a crown of a distal ring of portion 3580C.

FIG. 36 shows another embodiment of a stent. Stent 3680 includes portion 3680A, portion 3680C and portion 3680B between portion 3680A and portion 3680C. Stent 3680 is symmetric longitudinally in that portion 3680A and portion 3680C each have three rings of struts and six struts per ring. Portion 3680A and portion 3680C are similar to their counterparts described above with respect to stent 3580 (FIG. 35). Portion 3680B of stent 3680 includes four rings of 12 struts per ring. The struts have a ring width smaller than the ring width of the ring that make up portion 3680A or portion 3680C (e.g., half size). The struts have a smaller ring width than a ring width of the struts of portion 3580B of stent 3580. Adjacent struts of portion 3680B are 180 degrees out of phase and are connected between their crowns and valleys. A distal ring of portion 3680B is connected through every other crown to a valley of a proximal ring of portion 3680A. A proximal ring of portion 3680B is coupled and every other valley to a crown of a distal ring of portion 3680C.

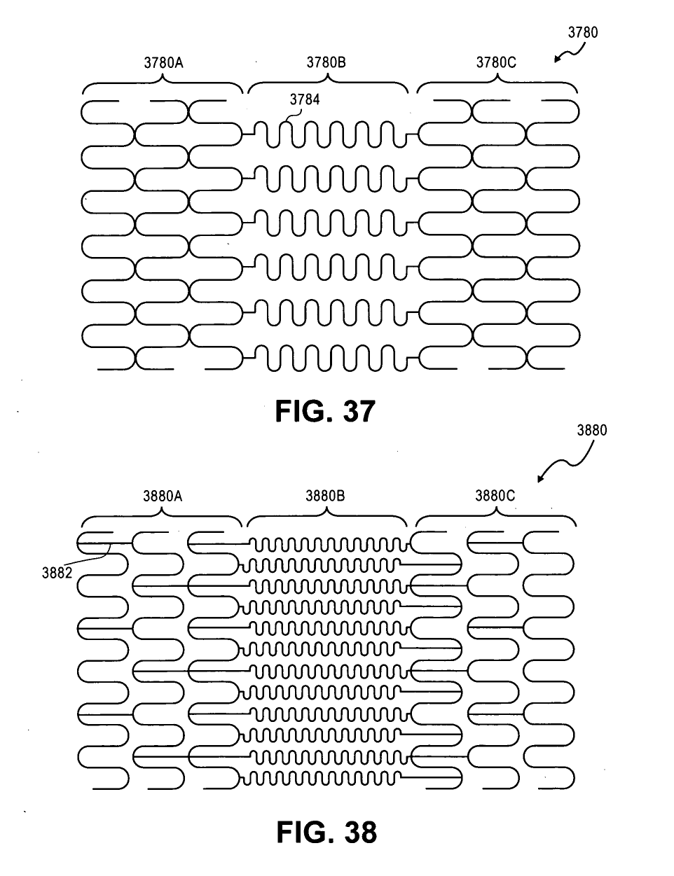

FIG. 37 shows another embodiment of a stent. Stent 3780 includes portion 3780A, portion 3780C and portion 3780B between portion 3780A and portion 3780C. Portion 3780A and portion 3780C each has three rings of six struts per ring. Adjacent rings of each of portion 3780A and portion 3780C are 180 degrees out of phase and the rings are connected between the crowns and valleys. The proximal ring of portion 3780A is also 180 degrees out of phase of the distal ring of portion 3780C.

Portion 3780B of stent 3780 includes six suspension elements, each suspension element connected between a valley of a proximal ring of distal portion 3780A and a crown of a distal ring of portion 3780C. Each suspension element has six undulations 3784. In one embodiment, portion 3780B is intended to be positioned in a blood vessel at a position including a vulnerable plaque.

FIG. 38 shows another embodiment of a stent. Stent 3880 includes portion 3880A, portion 3880B and portion 3880C between portion 3880A and portion 3880C. Portion 3880A and portion 3880C each has three rings of struts and six struts per ring. The rings in each portion are in phase and the rings are connected by axial links 3882 between their crowns. The proximal ring of portion 3880A is also in phase with the distal ring of portion 3880C.

Portion 3880B of stent 3880 has 12 suspension elements. With two suspension elements connected to each strut of a proximal ring of portion 3880A and a distal ring of portion 3880C, respectively. Each suspension element has 12 undulations. In one embodiment, portion 3880B is intended to be positioned in a blood vessel at a location including a vulnerable plaque.

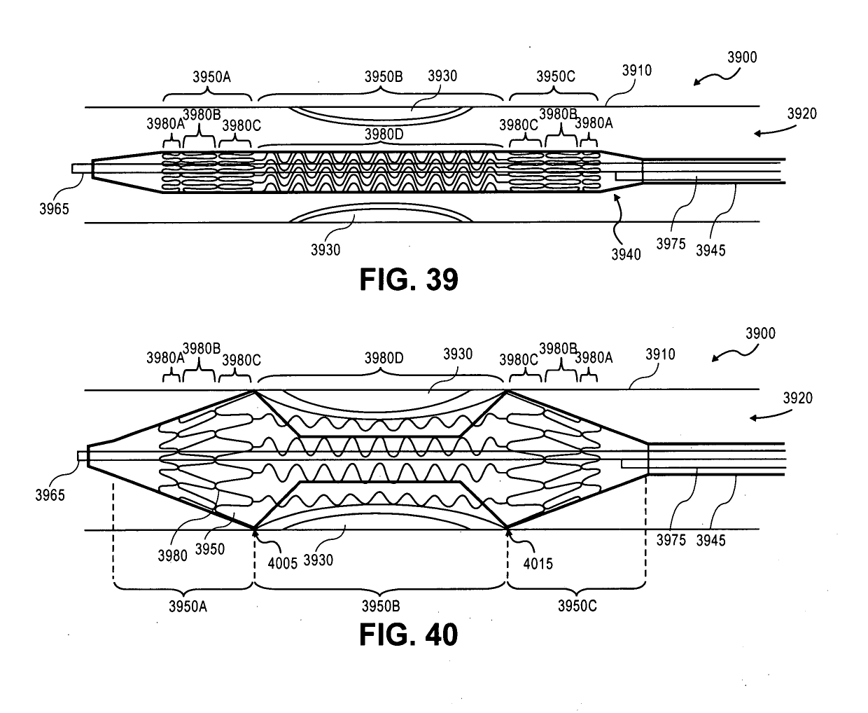

FIG. 39 shows another embodiment of a catheter assembly and a blood vessel including a vulnerable plaque. Blood vessel 3900 includes vessel wall 3910 having lumen 3920 therethrough. Vulnerable plaque 3930 is shown in blood vessel 3900. Vulnerable plaque 3930 modifies a lateral-cross-sectional shape of lumen 3920 from generally circular to irregular or oblong.

Disposed within lumen 3920 of blood vessel 3900 is catheter assembly 3940. Only a distal portion of catheter assembly 3940 is shown. Catheter assembly 3940 includes primary cannula or tubular member 3945. In one embodiment, primary cannula 3945 extends from a proximal end of catheter assembly 3940 intended to be external to a patient during a procedure, to a point proximal to a region of interest or treatment site within a patient. Representatively, catheter assembly 3940 may be percutaneously inserted via a femoral artery or a radial artery and advanced to a coronary artery. Catheter assembly 3940 includes guidewire cannula or tubular member 3965 disposed within a lumen of primary cannula 3945. Guidewire cannula 3965, in one embodiment, extends from a proximal end of catheter assembly 3940 so that catheter assembly 3940 may be advanced through a guidewire (not shown) in an over the wire (OTW) configuration. In another embodiment, guidewire cannula 3965 is present in only a distal portion of primary cannula 3945 and catheter assembly 3940 is advanced over a guidewire in a rapid exchange (RX) configuration.

Catheter assembly 3940 also includes balloon 3950. A proximal end (proximal skirt) of balloon 3950 is connected to a distal end of primary cannula 3945. A distal end (distal skirt) of balloon 3950 is connected to guidewire cannula 3965. In one embodiment, balloon 3950 has a working length longer than a length of vulnerable plaque 3930. In this manner, catheter assembly 3940 may be positioned within blood vessel 3900 such that a portion of balloon 3950 extends distal to (downstream) and proximal to (upstream) of vulnerable plaque 3930. FIG. 39 shows balloon 3950 having portion 3950A disposed downstream of vulnerable plaque 3930 and portion 3950C disposed upstream of vulnerable plaque 3930. Portion 3950B is disposed at a position within blood vessel 3900 including vulnerable plaque 3930. In FIG. 39, balloon 3950 is shown in a deflated or non-expanded state. In one embodiment, each of portion 3950A, portion 3950B and portion 3950C are expandable to a greater diameter. In another embodiment, only portion 3950A and portion 3950C are expandable.

Overlying a working length of balloon 3950 of catheter assembly 3940 is stent 3980. In one embodiment, the expansion characteristics of stent 3980 are varied across its length. Ways to modify the expansion characteristics of a stent include, but are not limited to, modifying a width and/or thickness of a strut or modifying a ring width. FIG. 39 shows stent 3980 having a variety of ring widths across its length. Struts of individual rings may also vary in width or thickness as desired. Referring to FIG. 39, stent 3980 includes portions 3980A, portions 3980B, portions 3980C and portions 3980D. In one embodiment, portions 3980A have a ring width that is less than portions 3980B which, in turn, has a ring width equal to or less than portions 3980C. In this manner, the expansion characteristics of stent 3980 tend to make portions 3980A harder to expand (open) than portions 3980B and portions 3980C (and possibly portions 3980B harder to expand than portions 3980C). In this embodiment, portion 3980D is the easiest portion to expand and has the least amount of mechanical strain making portion 3980D easier to stretch than any of the other portions.

Catheter assembly 3940 also includes inflation cannula or tubular member 3975. In one embodiment, inflation cannula extends from a primary portion of catheter assembly 3940 intended to be external to a patient during a procedure, beyond a distal end of primary cannula 3945 into balloon 3950. Inflation cannula 3975 extends through a lumen of primary cannula 3945. In an embodiment where a balloon includes separate portions, for example, portion 3950A and portion 3950C, separate inflation cannulas may be used to separately fill the portions.

FIG. 40 shows catheter assembly 3940 within blood vessel 3900 following the partial expansion of balloon 3950. In one embodiment where a working length of balloon 3950 includes portion 3950A, portion 3950B and portion 3950C, portion 3950A and portion 3950C initially expand to a greater extent than portion 3950B. The expansion characteristics of balloon 3950 may be controlled by selecting a material for the balloon or a method of manufacturing the balloon that allows portion 3950A and portion 3950C to expand at a reduced inflation pressure than an inflation pressure necessary to expand portion 3950B or to expand more rapidly than portion 3950B at the same inflation pressure. In terms of a method of making a balloon, representatively, portion 3950B may be made of ribbons of the polymer material having a greater thickness than ribbons used to form portion 3950A and portion 3950B; portion 3950B may have additional layers of polymer ribbon; or portion 3950B may have a smaller wind angle than portion 3950A and portion 3950C. In another embodiment, portion 3950B may be constructed so as not to expand or to minimally expand under the inflation pressure necessary to fully expand portion 3950A and portion 3950C.

As shown in FIG. 40, a proximal end of portion 3950A expands more rapidly than a distal portion. Similarly, a distal portion of portion 3950C expands more rapidly than a proximal portion. One way to achieve the proximal and distal end expansion is through the characteristics of stent 3980. For example, varying the width and/or thickness of a stent strut or a ring width of a stent strut, the expansion of stent 3980 may be modified. In the embodiment illustrated, the ring width of portions 3980A of stent 3980 are smaller than the right width of portions 3980B which inhibit a distal portion of portion 3950A of balloon 3950 from expanding and a proximal portion of portion 3950C from expanding. FIG. 40 shows portions 3980C expanding to a greater degree than portions 3980B and portions 3980A under the inflation pressure to achieve the partial expansion of balloon 3950. In this manner, portion 3950A of balloon 3950 is expanded such that a proximal end (illustrated at point 4005) contacts vessel wall 3910 of blood vessel 3900 and a distal end of portion 3950C contacts vessel wall 3910 of blood vessel 3900 (illustrated at point 4015). Expansion in this manner causes suspension elements in portion 3980D of stent 3980 to expand and stretch so that the suspension elements are suspended across vulnerable plaque between point 4005 and point 4015 gently contacting vulnerable plaque 3930. In this embodiment, the suspension elements in portion 3980D are expanded without a corresponding expansion of portion 3950B of balloon 3950.

As noted above, the struts of portions 3980A, portions 3980B and portions 3980C of stent 3980 expand at different rates with respect to an inflation pressure. As illustrated, portions 3980C expand at a lower inflation pressure than portions 3980B. Similarly, portions 3980B expand at a lower inflation pressure than portions 3980A. The variable rate of expansion of struts in portion 3980A, portion 3980B and portion 3980C inhibits any tendency of the strut to be pulled towards a location of the blood vessel including vulnerable plaque 3930.

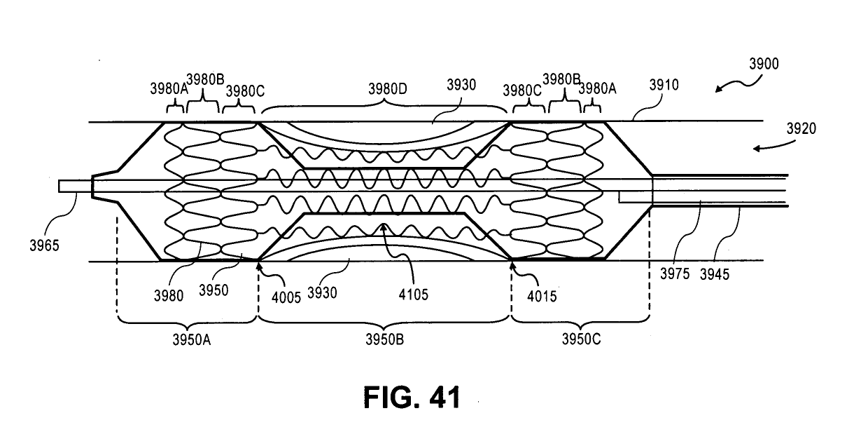

FIG. 41 shows catheter assembly 3940 after the further inflation of balloon 3950. As illustrated, balloon 3950 is expanded so that portion 3950A and portion 3950B bring stent 3980 into contact with the blood vessel wall. In other words, portion 3950A and portion 3950C are expanded to expand portions 3980A, portions 3980B and portions 3980C of stent 3980. A distal portion of portion 3950A and a proximal portion of portion 3950B are expanded to place the corresponding portions of stent 3980 in contact with wall 3910 of blood vessel 3900. The further expansion tends to deploy stent 3980 within blood vessel 3900. As balloon 3950 is expanded from the point shown in FIG. 40 to the point shown in FIG. 41, balloon 3950 tends to anchor stent 3980 against wall 3910 of blood vessel 3900 adjacent to vulnerable plaque 3930 by bringing stent 3980 into contact with the wall. The smaller ring width of portions 3980A tend to provide tension to stent 3980 until anchoring the blood vessel wall is sufficient.

Referring to FIG. 41, balloon 3950 is expanded to a desired diameter. In this manner, struts in portions 3980A, portions 3980B and portions 3980C are expanded to a desired position. Suspension elements of stent 3980 in portion 3980D sag slightly across a region of the blood vessel including vulnerable plaque 3930. In this manner, a portion of stent 3980 at approximately a mid-point of vulnerable plaque 3930 (illustrated at point 4105), has a smaller diameter than a portion of stent 3980 at point 4005 or at point 4015. In one embodiment, the suspension elements in portion 3980D of stent 3980 gently contact a fibrous cap of vulnerable plaque 3930 and provided stimulus for cap thickening and reinforcement. A degree of sag of portion 3980D can be controlled using parameters like an undulation amplitude of the suspension elements, width of the suspension elements as well as the relative stiffness of the struts in portions 3980A and portions 3980B.

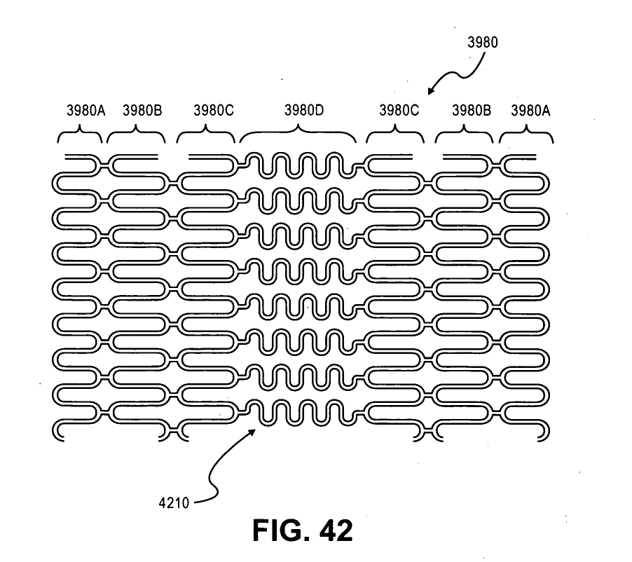

FIG. 42 shows a flattened view of an embodiment of stent 3980. Stent 3980 includes portions 3980A, portions 3980B, portions 3980C and portion 3980D. As illustrated in FIG. 42, a ring width of portions 3980A is less than a ring width of portions 3980B and portions 3980C. The shorter ring width tends to make portions 3980A more difficult to expand than portions 3980B or portions 3980C. Other ways to make portions 3980A more difficult to expand than the other portions include increasing the strut width or thickness or decreasing a strut length or some combination of the parameters.

As shown in FIG. 42, portions 3980A, portions 3980B and portions 3980C define rings each consisting of eight struts. The struts of adjacent rings are 180 degrees out of phase. The rings are connected by links 4283 at corresponding crowns and valleys (one link at each strut). In addition, the rings that make up portions 3980A, portions 3980B and portions 3980C at proximal and distal ends of stent 3980 are 180 degrees out of phase with their counterpart. FIG. 42 shows suspension elements in portion 3980D. In this embodiment, portion 3980D has eight suspension elements with the suspension elements intended to be equally spaced around a blood vessel. In another embodiment, a stent includes fewer suspension elements, possibly with a configuration such that suspension elements would be concentrated at an area of the blood vessel including a vulnerable plaque. The suspension elements include undulations that play a role in determining a sag to which portion 3980D will adopt when stent 3980 is deployed. Increasing the number of undulations will tend to decrease a sag. In FIG. 42, the suspension elements in portion 3980D are connected to respective crowns and valleys in rings 3980C at distal and proximal ends of stent 3980.

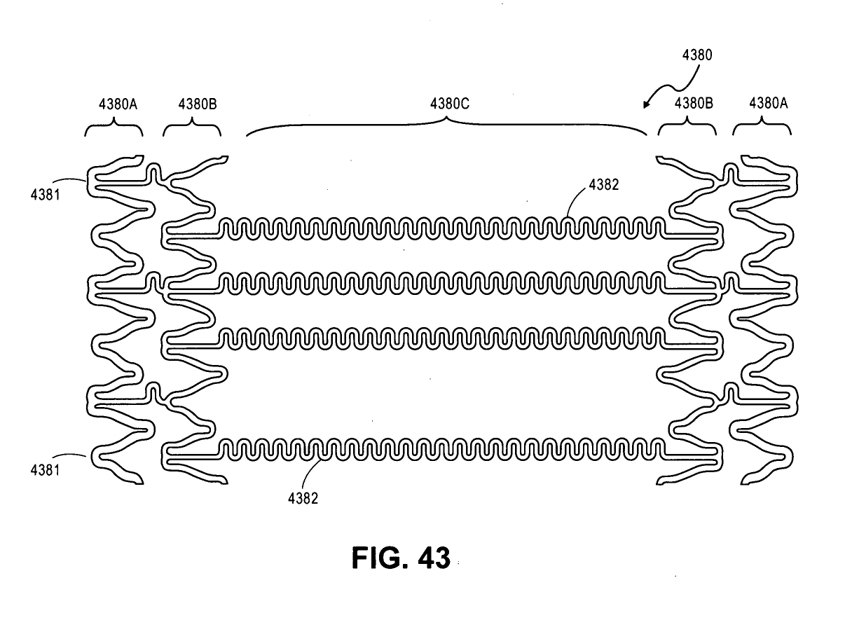

FIG. 43 shows another embodiment of a stent suitable, in one aspect, for use with the catheter assembly and method for reshaping a blood vessel lumen described in FIGS. 39-41 and the accompanying text. Referring to FIG. 43, stent 4380 is a modification of a VISION™ stent. Stent 4380 includes portions 4380A, portions 4380B and portion 4380C. Portions 4380A and portions 4380B define rings of struts that are intended to be deployed proximal and distal to a vulnerable plaque in a blood vessel. Portion 4380C has a number of suspension elements that are intended to be suspended across a vulnerable plaque.

Relative to stent 3980, portions 4380A and portions 4380B of stent 4380 each include six crowns 4381 as compared to the eight crowns of stent 3980. Stent 4380 also includes two ring portions (portion 4380A and portion 4380B) at its ends as compared to the three ring portions of stent 3980. In this embodiment, the struts of the rings are in phase and are connected at crowns 4381 by links 4383 disposed between every other strut. Stent 4380A has four suspension elements 4382 as compared to the eight suspension elements in stent 3980. Suspension elements 4382 have undulations similar to the undulations of the suspension elements of a VISION™ stent. FIG. 43 shows suspension elements 4382 concentrated in one portion (side) of the stent. In one embodiment, a higher density of suspension elements are intended to be oriented over a vulnerable plaque.

Additional comparison of stent 4380 to stent 3980 (see FIG. 42) shows that suspension elements 4382 are connected to the crowns of the struts in portion 4380B. In addition, the struts in portion 4380A and portion 4380B are similar to the struts of a VISION™ stent. The rings of portions 4380A, in one embodiment, have wider struts than the struts of the rings of portion 4380B. Finally, suspension elements 4382 are longer than the suspension elements shown in portion 3980D of stent 3980. It is appreciated that many combinations of the changes and attributes can be modified to optimize the performance of a stent for a given lesion.

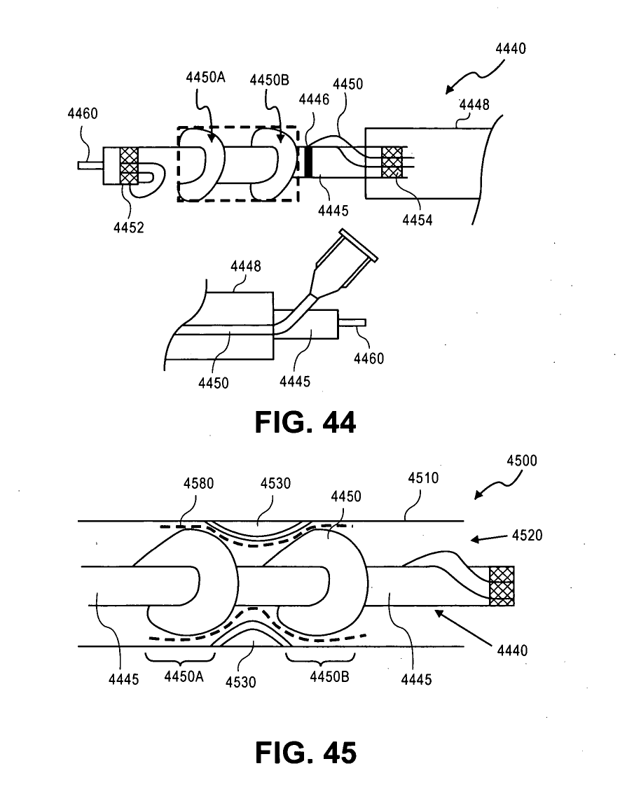

FIG. 44 illustrates another embodiment of a catheter assembly. In this embodiment, catheter assembly 4440 includes primary cannula 4445 that has a lumen of a sufficient size to accommodate a guidewire, such as guidewire 4460. In this manner, catheter assembly 4440 may be advanced over guidewire 4460 to a region of interest or a treatment site. In the embodiment shown, primary cannula 4445 extends from a proximal end of the catheter assembly intended to be exterior to a patient during a procedure to a distal end of a catheter assembly in an over the wire (OTW) configuration. In one embodiment, primary cannula 4445 has a length sufficient to be inserted into a patient at either a femoral or radial artery and advanced to a location within a coronary artery.

Primary cannula 4445 is a polymer material that may include markers to allow the cannula to be identified using fluoroscopic or angiographic techniques. For example, FIG. 45 shows marker 4446 that is, for example, a metal band (e.g., stainless steel or platinum) that may be detected by fluoroscopic or angiographic techniques.

In the embodiment shown in FIG. 44, catheter assembly 4440 includes balloon 4450 wrapped/spiraled at a distal end around primary cannula 4445. In the embodiment shown, balloon 4450 includes distal spiral 4450A and proximal spiral 4450B. Distal spiral 4450A is spaced from proximal spiral 4450B a distance greater than a projected length of a vulnerable plaque within a blood vessel (e.g., a distance between adjacent peaks of balloon 4450 is at least as large as a projected length of a vulnerable plaque). Distal spiral 4450A and proximal spiral 4450B may be configured to deploy a stent in a blood vessel around a vulnerable plaque. For reference, a typical vulnerable plaque may have a length on the order of three millimeters. Accordingly, a stent having a length on the order of six to seven millimeters would be sufficient to dispose a portion of the stent on either side of the vulnerable plaque. Thus, in one embodiment, distal spiral 4450A is placed approximately three millimeters from proximal spiral 4450B. A stent is shown in ghost lines to indicate the spacing of portions 4450A and portions 4450B.

In one embodiment, balloon 4450 may be connected to primary cannula 4445 at a distal end by strap 4452 and by strap 4454 at a portion of primary cannula 4445 intended to be positioned proximal to a region of interest. In one embodiment, a total inflatable size or length of a balloon is on the order of 10 mm to 20 mm. Representatively, the spacing of adjacent spirals is equivalent to approximately 50 percent of the total inflatable size of the balloon (e.g., 5 mm to 10 mm).

In one embodiment, balloon 4450 extends from a proximal end of catheter assembly 4440 intended to be external to a patient during a procedure to a distal portion of primary cannula 4445. In one embodiment, a material for balloon 4450 and its properties are selected so that the balloon expands along its entire length. Suitable materials for balloon 4450 include materials that will achieve expansion at inflation pressures on the order of six atmospheres or less. Suitable materials include, but are not limited to, PEBAX or ePTFE. In another embodiment, only the distal portion of balloon 4450 is intended to expand, notably a portion including spiral 4450A and spiral 4450B. Accordingly, the properties of balloon 4450 may be modified along its length making a portion proximal to spiral 4450A and spiral 4450B resistant to expansion at pressures less than six atmospheres.

In one embodiment, catheter assembly 4440 may be placed at a region of interest using a sheath that surrounds primary cannula 4445 and balloon 4450. FIG. 44 shows sheet 4448 overlying primary cannula 4445 and balloon 4450. A distal portion of primary cannula 4445 and balloon 4450 is exposed from the sheath, perhaps by retracting the sheath once catheter assembly 4440 is placed at the region of interest.

FIG. 45 shows an embodiment of a blood vessel including the catheter assembly of FIG. 44. FIG. 45 shows blood vessel 4500 including vessel wall 4510 and lumen 4520 therethrough. Disposed within blood vessel 4500 is vulnerable plaque 4530.

In the embodiment shown in FIG. 45, catheter assembly 4440 is placed such that distal spiral 4450A of balloon 4450 is positioned distal to vulnerable plaque 4530 and proximal spiral 4450B is placed proximal to vulnerable plaque 4430. FIG. 45 also shows stent 4580 overlying balloon 4450 across vulnerable plaque 4530.

In the embodiment shown in FIG. 45, balloon 4450 is in an expanded or inflated state. Spiral 4550A and spiral 4450B are expanded to anchor stent 4580 to the vessel wall at location distal and proximal to vulnerable plaque 4530. Stent 4580 is not expanded or is only partially expanded in the area of the blood vessel including vulnerable plaque 4530. In this manner, stent 4580 minimizes the expansion pressure on stent 4580 in the region including vulnerable plaque 4530. Thus, the possibility of rupturing vulnerable plaque 4530 is minimized. It is appreciated that as balloon 4450 is wrapped/spiraled around primary cannula 4445, a portion of balloon 4450 between distal spiral 4450A and proximal spiral 4450B may also expand. In one embodiment, catheter assembly 4440 may be positioned within blood vessel to minimize the possibility that an expanded portion of balloon 4450 in a region of blood vessel 4500 that includes vulnerable plaque 4530 actually contacts the vulnerable plaque. One way is to position the portion of balloon in the blood vessel on a side away from vulnerable plaque 4530.

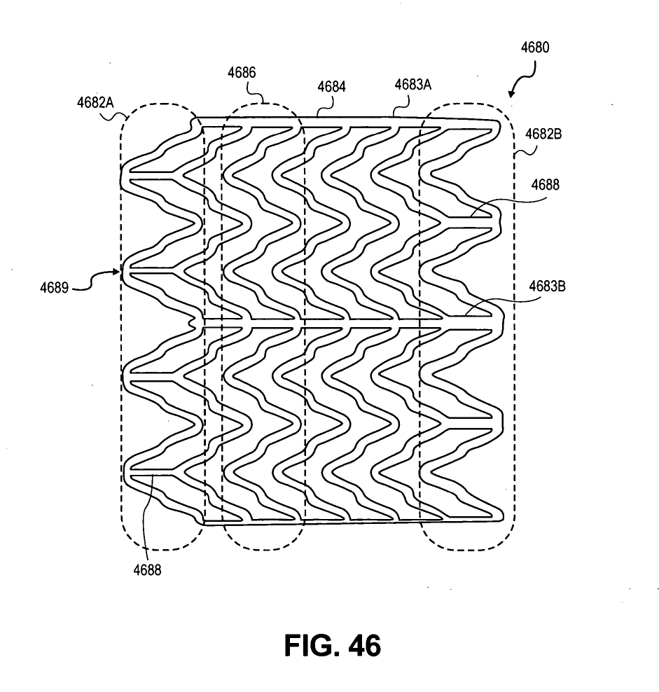

As noted above, one goal of deploying a stent around a vulnerable plaque is to stabilize or reinforce the plaque by way of the stent or by way of neointimal growth around the stent. One concern with a conventional metallic stent having metallic struts or suspension elements along the length of the stent is that a strut or suspension element could potentially rupture a fibrous cap of the vulnerable plaque either when the stent is deployed (e.g., while a balloon is inflated) or when a self-expanding metallic stent expands. Therefore, in another embodiment, a polymeric stent is contemplated. Such a stent may be one hundred percent polymer or a metal/polymer hybrid stent where, for example, the polymer portion of the stent is intended to be positioned at a location of the blood vessel including a vulnerable plaque. FIG. 46 shows an embodiment of a metal/polymer hybrid stent. Stent 4680 includes distal ring 4682A and proximal ring 4682B connected through axial link 4683A and axial link 4683B of a metal material. Although two axial links are shown, in another embodiment, one or three or more axial links may be utilized. Suitable metal materials for the metallic portion of stent 4680 include, but are not limited to, stainless steel or radiopaque metals such as platinum or gold. For self-expanding type stents, a shape memory material, such as a nickel-titanium alloy may be used as a metal material. A nickel-titanium-platinum alloy is one suitable metal material due to its generally high radiopacity. A representative thickness or the metallic portions of stent 4680 is on the order of 0.002 inches to 0.004 inches.

In the embodiment shown in FIG. 46, the metallic portions of stent 4680 (including ring 4682A, ring 4682B, axial link 4683A and axial link 4683B) are encapsulated in a polymer material. Similarly, stent 4680 includes a plurality of rings 4686 of polymeric struts disposed between ring 4682A and ring 4682B. Polymeric rings 4686 of stent 4680 are connected to proximal ring 4682A and distal ring 4682B through axial links 4688.

In one embodiment, a material for encapsulating the metal framework and for polymeric rings 4686 is non-biodegrable (e.g., non-absorbable) polymer material such as poly(butyleneterephalate) (PBT), poly(ethyleneterephalate) (PET) (e.g., DACRON), polypropylene, or expanded polytetrafluoroethylene (ePTFE).

One technique of fabricating a stent such as stent 4680 is to initially fabricate the metallic portion. Representatively, a metallic tube is fabricated into the ring and axial link portions using a laser. Following formation, the metallic portions are polished and etched. The resulting metallic portions (framework) of stent 4680 have, in one embodiment, a thickness on the order of 0.002 inches to 0.004 inches.

Following the formation of the metallic portion of stent 4680, the metallic portion is mounted onto a polymer tubing having a thickness on the order of 0.001 inches. The polymer tubing may be supported by a neckable metallic or polymeric mandrel or rod. A second polymer tubing having an inner diameter (ID) larger than the outside diameter (OD) of the metallic portion of stent 4680 and a thickness on the order of 0.001 inches to 0.002 inches is placed over the metallic portion of stent 4680. Shrink tubing may then be slid over the assembly. Heat is then applied to fuse the inner and outer polymer tubings while imbedding the metallic portions of the stent.

Following the fusion of the inner and outer polymeric tubings, a stent pattern may be fabricated in the fused polymer. In the proximal and distal crown area and where the metal axial links are located, the polymer is fabricated around the imbedded metal. Where there is no metal, a stent pattern is fabricated. Fabrication may be accomplished using a laser.

By using a radiopaque metal material for stent 4680, proximal ring 4682A and distal ring 4682B act as fluoroscopic markers where stent 4680 is placed in a blood vessel using, for example, angiographic or fluoroscopic techniques. Proximal ring 4682A and distal ring 4682B, in one embodiment, are intended to be positioned in a blood vessel on opposite sides of a vulnerable plaque (e.g., proximal and distal to a vulnerable plaque). The metallic portions of proximal ring 4682A and distal ring 4682B act as anchors against a vessel wall. The medial portion of stent 4680 including primarily polymeric rings 4686 may provide scaffolding to a vulnerable plaque while applying minimal force against the vulnerable plaque. The polymeric material will also tend to provide relatively low radial force in a vulnerable plaque area compared to conventional metal stents.

In another embodiment, stent 4680 may incorporate anti-proliferic, anti-thrombogenic, anti-inflammatory and/or anti-oxidative drugs into the polymer. For example, polymers such as PET and PBT have relatively low glass transition temperatures and are, therefore, susceptible to impregnation by such drugs using supercritical fluid impregnation techniques. In another embodiment, anti-proliferic, anti-thrombogenic, anti-inflammatory and/or anti-oxidative drugs may be coated on a surface of stent 4480. In a further embodiment, the polymer material of stent 4480A may be coated or carry cellular components such as endotheliol progenitor cells (EPC).

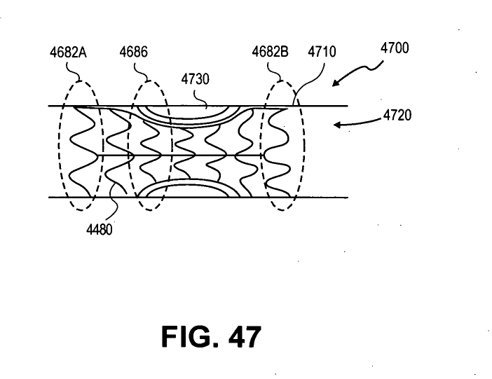

FIG. 47 shows stent 4680 disposed within a blood vessel. Blood vessel 4700 includes vessel wall 4710 having lumen 4720 therethrough. Disposed within lumen 4720 of blood vessel 4700 is vulnerable plaque 4730. Vulnerable plaque 4730 tends to modify a lateral-cross-sectional shape of lumen 4720 from circular to non-circular or oblong. Stent 4680 may be placed (anchored) within blood vessel 4700 by a balloon or as a self-expanding structure in a manner that a lateral-cross-sectional shape of lumen 4620 at vulnerable plaque 4730 is modified to a circular shape. This may be done, for example, by deploying stent 4680 using a balloon as described above. In another embodiment, a shape of lumen 4720 at vulnerable plaque may not be modified.

FIG. 47 shows stent 4680 having distal ring 4682A and proximal ring 4682B placed at a position within lumen 4720 of blood vessel 4700 distal and proximal to vulnerable plaque 4730, respectively. In an embodiment where stent 4680 includes metallic material as part of distal ring 4682A and proximal ring 4682B that is radiopaque, fluoroangiographic or fluoroscopic techniques may be used to position stent 4680.

In the above embodiment, a metal/polymer hybrid stent is described. In another embodiment, the stent may be formed solely as a polymeric stent, without any metal material. Still in another embodiment, a stent may be formed solely as a polymer material and then impregnated or coated with metal material in, for example, the distal or proximal rings. Deposition techniques, such as low temperature chemical vapor deposition may be employed to deposit metal on a polymer stent. Advantages of incorporating a metal material into a stent include the ability to use fluoroscopic techniques to position the stent and also that the metal material tends to improve the retention of a stent on a balloon during placement.

In terms of positioning a stent within a blood vessel percutaneously, there are two basic techniques. One technique utilizes a balloon with the stent disposed on an exterior of the working length of the balloon and expanding the balloon to expand and deploy the stent. An alternative technique is to construct a stent of expandable material and deliver the stent in a collapsed configuration generally enclosed within a sheath. Retracting the sheath allows the stent to expand and be deployed within the blood vessel. One suitable material for a self-expanding stent is a nickel-titanium alloy. Nickel-titanium alloy may have a shape memory of, for example, an expanded state. The shape may be minimized during positioning but return to its memorized shape on, for example, exposing the stent. Accordingly, in embodiment of a stent intended to be self-deployed (i.e., without the use of a balloon), a metal material such as a nickel-titanium alloy in an otherwise polymeric stent may be necessary to achieve the self expansion.

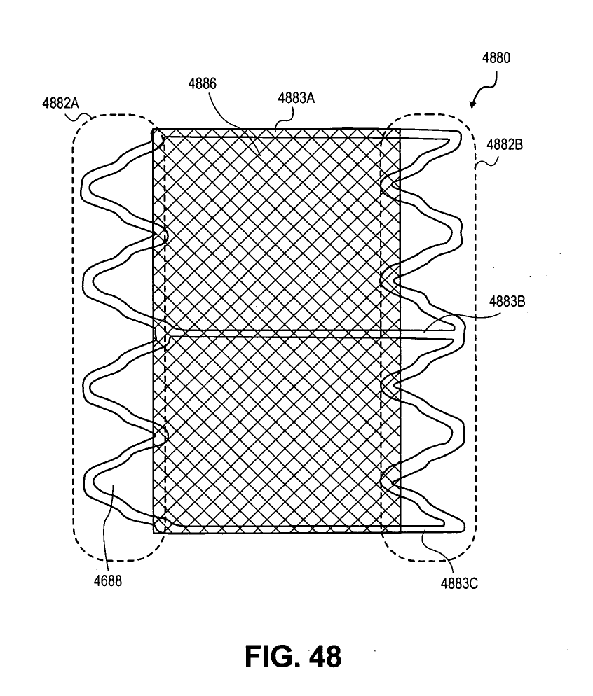

FIG. 48 shows a flattened view of another embodiment of a stent. Stent 4880 includes a metal frame defining ring 4882A and ring 4882B each of a plurality of struts. Ring 4882A and ring 4882B are connected through axial link 4883A, link 4883B and link 4883C. In other embodiments, fewer or more axial links may be employed. In one embodiment, the rings and links are a metal material. Suitable metal materials include, but are not limited to, stainless steel or radiopaque metals such as platinum or gold. Alternatively, suitable metal material may be a shape memory material such as a nickel-titanium alloy (e.g., a nickel-titanium-platinum alloy), particularly for self-expanding type stents. A representative thickness of the metallic portions (framework) of stent 4880 is on the order of 0.002 inches to 0.004 inches.

In the embodiment described with reference to FIG. 48, overlying the metal framework of stents 4880 is a polymer mesh or weave. FIG. 48 shows polymer mesh or weave 4886 overlying axial link 4883A, link 4883B and link 4883C between ring 4882A and ring 4882B. Suitable material for mesh or weave 4886 includes non-bioerodable material such as polypropylene mesh, such as PROLENE™, or a polyester fiber mesh such as MERSILENE™. PROLENE™ and MERSILENE™ are commercially available from Ethicon Products, a Johnson & Johnson Company. In another embodiment, fiber mesh 4886 may be an absorbable or bioerodable mesh, such as Polyglactin 910 knitted or woven mesh sold by Ethicon Products under the trade name VICRYL™.

As illustrated in FIG. 48, mesh or weave 4886 resembles a sheet that may be wrapped in one or more pieces around axial link 4883A, link 4883B and link 4883C. In one embodiment, a frame may be formed as described above with reference to FIG. 46 and the accompanying text and weave or mesh 4886 may be wrapped around the frame and glued or fused to the frame (e.g., glued or fused to axial link 4883A, link 4883B and/or link 4883C. In another embodiment, mesh or weave 4886 may be wrapped and connected at its ends (e.g., connected by the seams).

One advantage of a weave such as described as opposed to a film is that the weave should allow oxygen permeability.

In another embodiment, mesh or weave 4886 of stent 4880 may incorporate anti-proliferic, anti-thrombogenic, anti-inflammatory and/or anti-oxidative drugs into the mesh or weave material. The mesh or weave material may be impregnated using, for example, supercritical fluid impregnation. In another embodiment, mesh or weave 4886 may be coated with the drug. In still another embodiment, rather than a drug incorporated or coated on to a surface of the weave or mesh, a cellular component such as EPC cells may be incorporated or coated onto the mesh or weave. Finally, in the case of bioabsorbable polymer material; it is possible that the mesh or weave material may degrade via hydrolysis. Such degradation may be acceptable, for example, it is desired that the stent not cover the vulnerable plaque for an extended period of time. In another reports, bioabsorbable polymeric material has indicated inflammatory responses. To minimize such responses, a polymer mesh material could be coated or impregnated with a drug such as EVEROLIMUS™.

In a typical balloon deployment of a stent, inflation pressures greater than six atmospheres and approaching ten atmospheres or greater are generally required to inflate a balloon to a nominal dimension. A nominal dimension in this sense means a dimension equivalent to the inside diameter of a blood vessel. As noted above, vulnerable plaque is believed to be fairly fragile. High pressures may tend to promote the rupture of a vulnerable plaque. If conventional balloons are inflated at lower pressure to minimize rupture of the vulnerable plaque, balloon diameter may be difficult to predict or control. In addition, pressure below rated nominal pressure, the change in diameter with increasing pressure is generally quite rapid and uncontrollable. Finally, dilating the vulnerable plaque larger than desired could also prove to be detrimental in treating a vulnerable plaque with a stent.

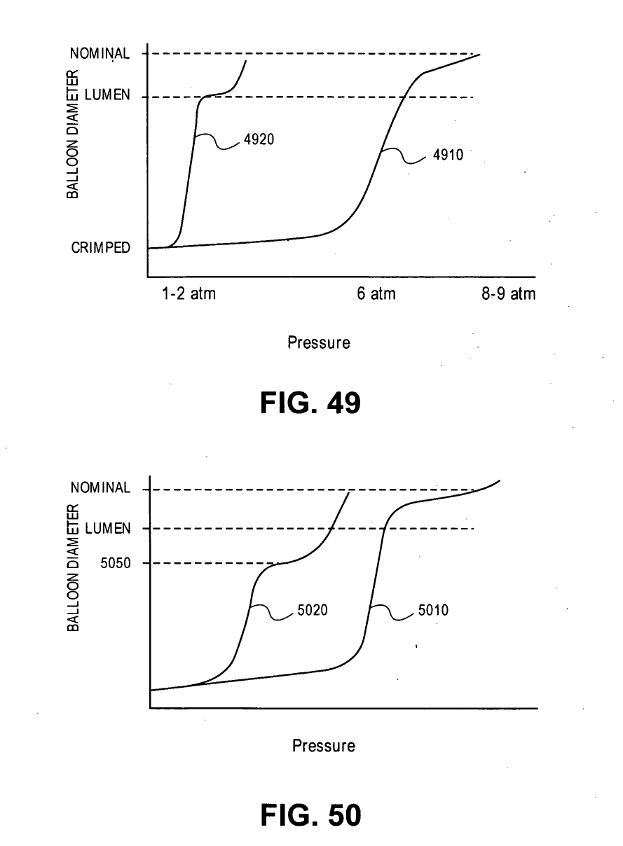

FIG. 49 presents a graphical representation of balloon diameter and inflation pressure. FIG. 49 shows the inflation pressure necessary to expand a balloon to an inner diameter of a blood vessel and to a nominal dimension, typically approximately ten percent larger than the inner diameter of the blood vessel where a stent is deployed. The larger increment accounts for some elastic recoil of the stent and/or the vessel. A working pressure range of a balloon is typically defined as the inflation pressure required to inflate a balloon to its nominal diameter. FIG. 49 shows curve 4910 of a conventional stent-deploying balloon of PEBAX or nylon. Curve 4910 shows that the balloon requires an inflation pressure of six atmospheres or more to inflate from a folded balloon configuration. During unfolding of the balloon, a conventional balloon expands rapidly to a dimension equivalent to the inner diameter of a blood vessel. Once fully unfolded, to increase the diameter beyond the diameter of a blood vessel to reach a nominal diameter of, for example, ten percent greater than the inner diameter of a blood vessel, the change in diameter with increasing pressure is more gradual since increasing pressure is accompanied by distending of the balloon material (less compliant portion of the compliance curve). Unfolding of a folded balloon requires lower pressure than distending the fully unfolded balloon to a larger diameter.

In one embodiment, a balloon material is selected that has a property that will demonstrate a relatively flat portion of compliance at fairly low working pressures. In terms of compliance, curve 4910 of FIG. 49 tends to show that a conventional balloon becomes less compliant at about ten atmospheres.

In one embodiment, a material for an inflation balloon of a catheter assembly has a property such that it has a relatively flat portion of compliance (e.g., is less compliant) at fairly low working pressures (nominal of one to two atmospheres, quarter size of four to five atmospheres). Thus, the material and size of the balloon is selected such that it can be inflated to a nominal diameter at low pressures and becomes less compliant at a nominal diameter.

Referring to FIG. 49, a suitable balloon may have an inflation representation of curve 4920 which shows that a balloon may be inflated to an inner diameter of a lumen (e.g., a diameter at a point having a vulnerable plaque) at low pressures (e.g., one to two atmospheres). A suitable balloon is non-distending in that the balloon unfolds without balloon material stretching. In the example of using the balloon to deploy a stent, an expansion of the stent is due to the balloon going from a folded to an unfolded state (“geometric compliance”).

Once reaching the inflation diameter equivalent to an inner diameter of a lumen by unfolding of a folded balloon, the balloon becomes relatively less compliant and significantly greater pressure (e.g., four to five atmospheres) is required to further expand the balloon. When a conventional balloon and the balloon having the expansion property illustrated in curve 4920 unfold to a fully unfolded state, the balloon become less compliant. By appropriately sizing the balloon having the expansion property illustrated in curve 4920, the balloon unfolds to a larger diameter more quickly at lower pressure. Representatively, a balloon having the expansion property illustrated in curve 4920 may have a starting diameter that is about 10 percent to 40 percent larger than a diameter of a conventional balloon that is fully unfolded (e.g., about 0.5 mm or larger diameter). For example, to deliver a stent in a blood vessel over an area including a vulnerable plaque, a target vessel inner diameter is, for example, 2.75 mm. A balloon having the expansion characteristics illustrated in curve 4920 expands to an inflated outer diameter in a fully unfolded state of 2.7 mm of about 1 atm. A conventional balloon might expand to a fully unfolded diameter of about 2.35 mm at about 6 atm (about 15 percent less than a balloon having an expansion property illustrated in curve 4920).

Suitable materials for a balloon having a relatively flat portion of a compliance curve at fairly low working pressures, particularly inflation pressures less than two atmospheres and preferably between one to two atmospheres. Suitable materials include polymer materials having a two percent secant modulus (ASTM D882) less than 60,000 PSI or flexural modulus (ASTM D790) less than 36,000 PSI. A suitable material may be radiation cross-linkable and preferably may be thermally or adhesively bonded to commonly used catheter shaft materials such as polyolefin, polyamide or block polyamide. Examples of suitable materials for an inflation balloon include, but are not limited to, copolyamides such as PEBAX from Atofina, or their blends, and polyamides. Polyolefins, modified polyolefins, co-polymers polyolefins and metallocene polyolefins may also be suitable. Specific examples include ethylene vinyl acetate (EVA) such as ESCORENE™ from ExxonMobil Chemical Company or BYNEL™ from Dupont Packaging Industrial Polymers; ethylene methyl acrylate (EMAC) such as ELVALOY™ from Dupont Packaging & Industrial Polymers or OPTEMA™ from ExxonMobil Chemical Company; ENGAGE™ polymer from Dupont Dow Elastomers; and ethylene acrylic acid (EEA) co-polymer such as PRIMACOR™ from Dow Plastics.

To form a folded balloon such as described, the polymer may be extruded into a tubing. For polyolefins, modified polyolefins, co-polymers of polyolefins and metallocene polyolefins, the tubing may be irradiated with an appropriate dose (e.g., typically about 20-50 MRad) to be blown into a given size balloon . Such balloon should be expected to have an average rupture pressure of at least ten atmospheres preferably at least fifteen atmospheres with a flat portion of the compliance curve at fairly low working pressures (e.g., nominal at one to two atmospheres, quarter size at four to five atmospheres). Quarter size refers to size of the balloon where diameter reaches nominal plus 0.25 mm (a quarter mm).

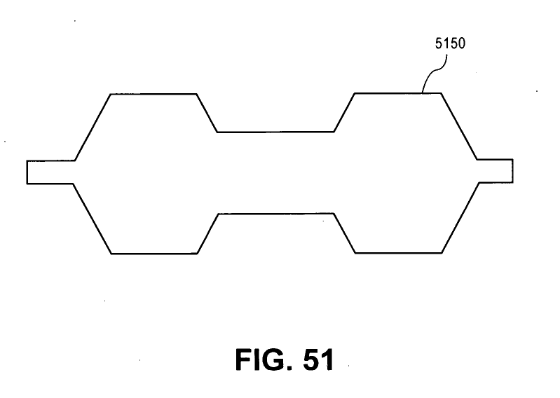

As noted above, a vulnerable plaque is perceived to be fairly fragile. Thus, there may be a concern about contacting the vulnerable plaque with a stent or a balloon. Thus, in another embodiment, an expansion property of a balloon may be selected and modified such that the balloon has a relatively flat compliance (e.g., non-compliance) at an expanded diameter less than the inner diameter of a blood vessel. FIG. 50 shows a graphical representation of a balloon expansion. FIG. 50 shows inflation curve 5010 of a conventional inflation balloon (e.g., a stent-deploying balloon) and curve 5020 according to this embodiment. Inflation curve 5010 is similar to curve 4910 described above with reference to FIG. 49. Referring to curve 5020, in this embodiment, at an inflation pressure of approximately three atmospheres, the balloon expands rapidly to a diameter that is less (e.g., 20-30 percent less) than an inner diameter of a blood vessel. This diameter is indicated at point 5050. At that point, with increasing pressure, the balloon becomes generally non-compliant. As the pressure approaches six atmospheres, the balloon may then expand more rapidly to a diameter equivalent to the inner diameter of a blood vessel and possibly greater. The non-compliance at a diameter less than an inner diameter of a blood vessel allows the increase to an inner diameter of the blood vessel to be more gradual with increasing pressure (e.g., the diameter will slowly grow until a desired lumen shape and size are reached). One configuration of a suitable balloon having inflation curve 5020 is a balloon that has at least two sections having at least two different diameters at low inflation pressure. FIG. 51 shows balloon 5150 having a dog-bone or dumb-bell shape.

In the preceding detailed description, reference is made to specific embodiments thereof. It will, however, be evident that various modifications and changes may be made thereto without departing from the broader spirit and scope of the following claims. The specification and drawings are, accordingly, to be regarded in an illustrative rather than a restrictive sense.

What is claimed is:

1. A method comprising:
introducing an expandable body into a blood vessel at a point coextensive with a vulnerable plaque lesion; and
expanding the expandable body from a first diameter to a different second diameter sufficient to modify the shape of an inner diameter of the blood vessel at the point coextensive with the lesion without rupturing the lesion.

2. The method of claim 1, wherein one of a shape of an inner diameter of the blood vessel is modified from a non-circular shape to a shape approaching that of a circle.

3. The method of claim 1, further comprising at least one of:
introducing a detectable agent into the blood vessel and wherein expanding comprises expanding the expandable body until the detectable agent is not detectable at a point between the expandable body and the lesion;
introducing a stent into the vessel on the expandable body and deploying the stent within the vessel.

4. The method of claim 3, wherein the method further comprises introducing a detectable agent and wherein at least one of:
the detectable agent is a radiopaque contrast agent; and
introducing a second expandable body distal to the lesion and prior to introducing the detectable agent, expanding the second expandable body to a dimension sufficient to occlude the blood vessel.

5. The method of claim 3, wherein the stent comprises a first expansion characteristic and a second different expansion characteristic, wherein the method comprises:
aligning that portion of the stent with the first expansion characteristic within the vessel corresponding to the point co-extensive with the lesion;
aligning that portion of the stent with the second expansion characteristic within the vessel adjacent to the lesion; and
expanding that portion of the stent with the second expansion characteristic to a diameter corresponding to an interior diameter of the vessel.

6. The method of claim 5, wherein that portion of the stent with the second expansion characteristic is expanded before that portion of the stent with the first expansion characteristic.

7. The method of claim 5, further comprising:
introducing a third expandable body proximal to the first expandable body.

8. A method comprising:
introducing a catheter comprising an expandable body having a first portion bounded by a second portion and a third portion into a blood vessel comprising a vulnerable plaque lesion, wherein the first portion is introduced at a point coextensive with a vulnerable plaque lesion; and
expanding the second portion and the third portion of the expandable body to a diameter greater than a diameter of the first portion.

9. The method of claim 8, wherein one of:
expanding comprises expanding the first portion of the expandable body from a first diameter to a different second diameter sufficient to modify the shape of an inner diameter of the blood vessel and retain a comparable perimeter; and
expanding the first portion independent of the expansion of the second portion and the third portion.

10. The method of claim 8, further comprising:
introducing a stent into the vessel on the expandable body; and
deploying the stent within the vessel.

11. The method of claim 10, wherein the second portion of the expandable body is proximal to the first portion and each of the second portion and the third portion of the expandable body comprises a proximal section and a distal section, and expanding comprises:
expanding the distal section of the second portion of the expandable body at a faster rate than the proximal section; and
expanding the proximal section of the third portion of the expandable body at a faster rate than the distal section.

12. The method of claim 11, wherein the stent comprises a first expansion characteristic and a second different expansion characteristic, wherein the method comprises:
aligning that portion of the stent with the first expansion characteristic within the vessel corresponding to the point coextensive with the lesion;
aligning that portion of the stent with the second expansion characteristic within the vessel adjacent to the lesion; and
expanding that portion of the stent with the second expansion characteristic to a diameter corresponding to an interior diameter of the vessel.

13. A method comprising:
introducing a catheter comprising an expandable body having a first portion bounded by a second portion and a third portion into a blood vessel comprising a vulnerable plaque lesion, wherein the first portion is introduced at a point coextensive with a vulnerable plaque lesion;
introducing a stent on the expandable body, the stent comprising a portion overlying the first portion of the expandable body; and
expanding the second portion and the third portion of the expandable body to a diameter greater than a diameter of the first portion and sufficient to introduce a tensile stress on the portion of the stent overlying the first portion of the expandable body.

14. The method of claim 13, wherein each of the second portion and the third portion comprise a working length including a proximal end and a distal end,
wherein the stent overlies the second portion and the third portion,
wherein introducing the catheter comprises introducing the second portion at a point distal to the lesion and the first portion at a point proximal to the lesion, and
wherein expanding comprises expanding the proximal end of the second portion of the expandable body to a diameter different than the distal end, and expanding the distal end of the third portion to a diameter different than the proximal end.

15. The method of claim 14, wherein a modification in an expansion characteristic of the stent across its length achieves an expansion difference between the proximal portion and the distal portion of each of the second portion and the third portion of the expandable body.

16. The method of claim 13, wherein expanding increases a diameter of the portion of the stent overlying the first portion of the expandable body.

17. The method of claim 16, wherein the diameter of the portion of the stent overlying the first portion of the expandable body comprises a proximal portion and a distal portion having a diameter greater than a medial portion.

18. The method of claim 17, wherein, following expanding, the medial portion of the stent contacts the lesion.

19. The method claim 14, wherein the second portion is at a point in the blood vessel distal to the lesion and the third portion is at a point in the blood vessel proximal to the lesion and expanding the second portion and the third portion to different diameters at respective proximal and distal ends comprises an initial expanding, the method further comprising:
following the initial expanding, subsequently expanding the distal end of the second portion and the proximal end of the third portion to a diameter sufficient to anchor the stent to the blood vessel.

20. A method comprising:
introducing a catheter comprising an expandable body having a working length into a blood vessel comprising a vulnerable plaque lesion, wherein the expandable body is at a point coextensive with a vulnerable plaque lesion; and
expanding the expandable body to a variable diameter along the working length such that at a point coextensive with the lesion the working length has a smallest diameter.

21. The method of claim 20, wherein at least one of:
the working length of the expandable body has a variable expansion property;
the working length of the expandable body is greater than a length dimension of the lesion within the blood vessel and the expandable body is at a point in the blood vessel proximal and distal to the lesion; and
a stent overlies the working length of the expandable body and an expansion property of the stent contributes to the variable diameter to which the expandable body is expanded.

22. An apparatus comprising:
a cannula having a dimension suitable for insertion into a blood vessel and an expandable body coupled thereto, the expandable body comprising a first outer diameter suitable for insertion through the blood vessel and a second outer diameter greater than the first diameter and having a maximum dimension to modify the shape of an inner diameter of the blood vessel and retain a similar perimeter.

23. The apparatus of claim 22, wherein the expandable body comprises a balloon of a material having a compliance greater than a compliance of an angioplasty balloon.

24. The apparatus of claim 23, wherein the balloon comprises a nominal pressure of less than five atmospheres.

25. The apparatus of claim 22, wherein the expandable body comprises a first expandable body, the apparatus further comprising a second expandable body coupled to the cannula at a point distal to the first expandable body, wherein the second expandable body comprises a first outer diameter suitable for insertion through the blood vessel and a second outer diameter greater than the second diameter of the first expandable body.

26. The apparatus of claim 25, wherein the cannula has a length suitable to locate the second expandable body in a blood vessel beyond a vulnerable plaque lesion.

27. The apparatus of claim 26, wherein at least one of:
the first expandable body has a length dimension corresponding to a length dimension of a vulnerable plaque lesion; and
the first expandable body and the second expandable body each comprise a balloon and the balloon of the first expandable body comprises a material having a compliance greater than a compliance of a material of the second expandable body.

28. The apparatus of claim 27, wherein the compliance of the material of the second expandable body is similar to a compliance of an angioplasty balloon,
wherein each of the first portion, the second portion, and the third portion of the expandable body comprises an expansion lumen and an expansion lumen of the first portion is isolated from an expansion lumen of the second portion and the third portion.

29. The apparatus of claim 22, wherein at a distal portion of the cannula, the expandable body is wound around the cannula such that a spacing between adjacent windings is at least as large as a projected length of a vulnerable plaque within the blood vessel.

30. A kit comprising:
a cannula having a dimension suitable for insertion into a blood vessel and comprising an expandable body coupled thereto, the expandable body comprising a first outer diameter suitable for insertion through the blood vessel and a second outer diameter greater than the first diameter and the second diameter has a maximum dimension to modify the shape of an inner diameter of the blood vessel and retain a same perimeter; and
a stent having a diameter suitable for deployment on the expandable body through a blood vessel.

31. The kit of claim 30, wherein the expandable body comprises a first expandable body, the apparatus further comprising a second expandable body coupled to the cannula at a point distal to the first expandable body, wherein the second expandable body comprises a first outer diameter suitable for insertion through the blood vessel and a second outer diameter greater than the second diameter of the first expandable body and wherein the stent comprises a length corresponding to a working length of the first expandable body.

32. The kit of claim 31, wherein the stent comprises a first portion having a length corresponding to a length of the first expandable body and second portion extending over a portion of the second expandable body, wherein the second portion has an expansion characteristic different from an expansion characteristic of the first portion.

33. The kit of claim 32, wherein the expansion characteristic of the second portion of the stent has a greater tendency to expand than the expansion characteristic of the first portion.

34. The kit of claim 33, wherein the first expandable body and the second expandable body each comprise a balloon and the balloon of the first expandable body comprises a material having a compliance greater than a compliance of a material of the second expandable body.

35. The kit of claim 30, wherein at a distal portion of the cannula, the expandable body is spiraled around the cannula such that a spacing between adjacent peaks of the expandable body is at least as large as a projected length of a vulnerable plaque within the blood vessel.

36. An apparatus comprising:
an expandable framework having an expanded diameter suitable for placement in a blood vessel and comprising a first end and a second end and a polymeric material disposed between the first end and the second end and defining a lumen therethrough.

37. The apparatus of claim 36, wherein the framework comprises a plurality of circumferentially disposed rings disposed a distance from one another, wherein each of the plurality of rings comprises a plurality of struts.

38. The apparatus of claim 37, wherein at least one of:
the first end comprises a first circumferentially disposed ring comprising a metal material;
the second end comprises a second circumferentially disposed ring comprising a metal material;
the polymeric material encapsulates the metal material; and
the polymeric material is patterned into a framework comprising at least one of struts and suspension elements.

39. The apparatus of claim 36, wherein the polymeric material comprises a non-bioerodable polymeric material.

40. The apparatus of claim 39, wherein a portion of the polymeric material comprises one of a drug and a cellular component.

41. The apparatus of claim 40, wherein the one of the drug and the cellular component is coated on a surface of the polymeric material.

42. The apparatus of claim 37, wherein the polymeric material comprises a mesh or weave overlying the metal material.

43. An apparatus comprising:
an expandable body having a diameter suitable for insertion into a blood vessel and capable of being modified from a first folded diameter to a second larger unfolded diameter in response to an inflation pressure less than two atmospheres and, following modification, being non-compliant at an inflation pressure less than two atmospheres.

44. The apparatus of claim 43, wherein at least one of:
in an unfolded state a diameter of the expandable body approximates a diameter of the blood vessel; and
the expandable body comprises a polymer having one of a two percent secant modulus less than 60,000 psi or a flexural modulus less than 36,000 psi.

45. The apparatus of claim 43, further comprising a cannula shaft wherein the expandable body is coupled to the cannula shaft.

46. An apparatus comprising:
an expandable body having a diameter suitable for insertion into a blood vessel and capable of being modified from a first diameter to a second larger diameter in response to an inflation pressure, wherein the second diameter is less than an inside diameter of the blood vessel and, following modification, being less compliant at an increased inflation pressure.

47. The apparatus of claim 46, further comprising a cannula shaft wherein the expandable body is coupled to the cannula shaft.

48. An apparatus comprising:
a balloon expandable intralumenal framework comprising a first end and a second end defining a length dimension longer than a length of a vulnerable plaque, the framework comprising axially-oriented anchor portions at the first end and the second end capable of anchoring to a blood vessel and supporting a medial portion between the ends without anchoring the medial portion to the blood vessel.

49. The apparatus of claim 48, wherein at least one of:
in an expanded state, the first end has a first diameter and the medial portion has a variable diameter that is less than or equal to the first diameter across its length;
an expansion of the medial portion depends on the expansion of the anchor portions; and
an expansion of the anchor portions from a first diameter to a larger second diameter increases a tensile strain on the medial portion.
